(19) United States
(12) Patent Application Publication
(10) Pub. No.: US 2009/0215835 A1
(43) Pub. Date: Aug. 27, 2009

(54) TREATMENT OF CANCER WITH SORAFENIB

(76) Inventor: Scott Wilhelm, Morristown, NJ (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/092,024

(22) PCT Filed: Oct. 31, 2006
(86) PCT No.: PCT/US2006/042367

§ 371 Date: Oct. 17, 2008

Related U.S. Application Data
(60) Provisional application No. 60/731,597, filed on Oct. 31, 2005.

(51) Int. Cl. A61K 31/44; A61P 35/00

(52) U.S. Cl. 514/350

(57) ABSTRACT

The present invention provides compositions and methods for treating specific cancers with effective amounts of sorafenib.

### DESCRIPTION OF THE INVENTION

Cancer is a class of diseases characterized by two heritable properties: (1) uncontrolled cell division and (2) the ability of these cells to invade other tissues, either by direct growth into adjacent tissue (invasion) or by migration of cells to distant sites (metastasis). The hyper-proliferative properties initially give rise to a tumor or neoplasm. A tumor is only considered a cancer when its cells acquire the ability to invade surrounding tissues, e.g., by breaking loose and entering the blood or lymph systems, or by forming secondary tumors at other sites in the body. The unregulated growth is caused by damaged DNA, resulting in mutations to vital genes that control cell division, the cell cycle, among other functions. One or more of these mutations, which can be inherited or acquired, can lead to uncontrolled cell division and cancer.

Cancers can be classified according to the tissue and cell type from which they arise. Cancers developing from epithelial cells are called carcinomas, and those from connective and muscle cells are called sarcomas. Additional cancers include those arising from hematopoietic cells (e.g., leukemia) and cancers of the nervous system.

In general, cancers appear to arise during a process in which an initial population of abnormal cells evolve into more aberrant cells through successive cycles of mutation and selection. More than 100 different genes have been identified which, when mutant, result in cancer. These so-called cancer-critical genes fall into two broad classes: oncogenes and tumor suppressor genes. Many cancer-critical genes play a role in the regulation of cell divisions, a highly complicated process involving multiple and parallel pathways. These include growth factors, cytokines, hormones, etc.

Cancer can cause many different symptoms, depending on the site and character of the malignancy and whether there is metastasis. A definitive diagnosis usually requires the microscopic examination of tissue obtained by biopsy. Once diagnosed, cancer is usually treated with surgery, chemotherapy and/or radiation.

If untreated, most cancers eventually cause death. Cancer is one of the leading causes of death in developed countries. It is estimated by the National Cancer Institute that approximately 9.8 million Americans were alive in January 2001 with a history of cancer. About 1,372,910 new cases of cancer were expected to be diagnosed in 2005, alone. In 2005, almost 600,000 Americans died of cancer, about 1 out of every 4 deaths. Many forms of cancer are associated with environmental factors, which may be avoidable. Smoking tobacco leads to more cancers than any other environmental factor.

The present invention provides methods of treating specific cancers in listed in Table 1, comprising, e.g., comprising administering to a subject in need thereof an effective amount of sorafenib, wherein the cancer is treated.

The phrase “effective amount” indicates the amount of sorafenib which is effective to treat any symptom or aspect of the specific cancer. Effective amounts can be determined routinely. Further guidance on dosages and administration regimens is provided below.

The term “treating” is used conventionally, e.g., the management or care of a subject for the purpose of combating, alleviating, reducing, relieving, improving, etc., one or more of the symptoms associated with a specific cancer mentioned herein in Table 1. Administering effective amounts of sorafenib can treat one or more aspects of the cancer disease, including, but not limited to, causing tumor regression; causing cell death; causing apoptosis; causing necrosis; inhibiting cell proliferation; inhibiting tumor growth; inhibiting tumor metastasis; inhibiting tumor migration; inhibiting tumor invasion; reducing disease progression; stabilizing the disease; reducing or inhibiting angiogenesis; prolonging patient survival; enhancing patient's quality of life; reducing adverse symptoms associated with cancer; and reducing the frequency, severity, intensity, and/or duration of any of the aforerentioned aspects.

Any of the listed cancers can be treated in accordance of the present invention, irrespective of the cause of the cancer, and irrespective of the genetic lesions associated with it (see, e.g., Atlas of Genetics and Cytogenetics in Oncology and Haematology on the worldwide web at infobiogen.fr/services/chromcancer/ for an atlas of genes involved in cancer). Table 1 is a list of cancers that be treated with sorafenib.

Cancers which can be treated include, e.g., cancers which are primary; which arise from a primary tumor at a secondary metastatic site; which have been treated by surgery (e.g., entirely removed, surgical resection, etc); which have been treated by chemotherapy, radiation, radiofrequency ablation, and/or any other adjunct to drug therapy; which have acquired drug-resistance; which are refractory to a chemotherapeutic agent.

Any subject can be in accordance with the present invention, including, e.g., mammals, such as rats, mice, dogs, cats, horses, cows, goats, sheep, monkeys, and humans.

The term “sorafenib” as used herein refers to the tosylate salt of the compound N-[4-chloro-3-(trifluoromethyl)phenyl]-N′-{4-[2-carbamoyl-1-oxo-(4-pyridyloxy)]phenyl}urea of the formula I below including all polymorphs, hydrates, solvates or combinations thereof. Formula I is as follows:

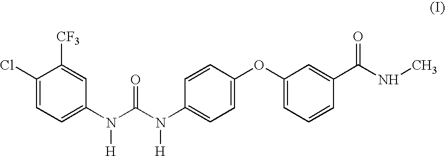

The compound N-[4-chloro-3-(trifluoromethyl)phenyl]-N′-{4-[2-carbamoyl-1-oxo-(4-pyridyloxy)]phenyl}urea can be prepared by the following multistep procedure:

### Step 1: Preparation of 4-chloro-2-pyridinecarboxamide

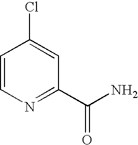

To a stirred mixture of methyl 4-chloro-2-pyridinecarboxylate hydrochloride (1.0 g, 4.81 mmol) dissolved in conc. aqueous ammonia (32 mL) is added ammonium chloride (96.2 mg, 1.8 mmol, 0.37 equiv.), and the heterogeneous reaction mixture is stirred at ambient temperature for 16 h. The reaction mixture is poured into EtOAc (500 mL) and water (300 mL). The organic layer is washed with water (2×300 mL) and a saturated NaCl solution (1×300 mL), dried (MgSO₄), concentrated in vacuo to give 4-chloro-2-pyridinecarboxamide as a beige solid (604.3 mg, 80.3%): TLC (50% EtOAc/hexane) R_{f}0.20; ¹H-NMR (DMSO-d₆) δ 8.61 (d, J=5.4 Hz, 1H), 8.20 (broad s, 1H), 8.02 (d, J=1.8 Hz, 1H), 7.81 (broad s, 1H), 7.76 to 7.73 (m, 1H).

### Step 2: Preparation of 4-(4-aminophenoxy)-2-pyridinecarboxamide

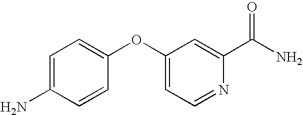

To 4-aminophenol (418 mg, 3.83 mmol) in anh DMF (7.7 mL) is added potassium tert-butoxide (447 mg, 3.98 mmol, 1.04 equiv.) in one portion. The reaction mixture is stirred at room temperature for 2 h, and a solution of 4-chloro-2-pyridinecarboxamide (600 mg, 3.83 mmol, 1.0 equiv.) in anh DMF (4 mL) is then added. The reaction mixture is stirred at 80° C. for 3 days and poured into a mixture of EtOAc and a saturated NaCl solution. The organic layer is sequentially washed with a saturated NH₄Cl solution then a saturated NaCl solution, dried (MgSO₄), and concentrated under reduced pressure. The crude product is purified using MPLC chromatography (Biotage®; gradient from 100% EtOAc to followed by 10% MeOH/50% EtOAc/40% hexane) to give the 4-chloro-5-trifluoromethylaniline as a brown solid (510 mg, 58%). ¹H-NMR (DMSO-d₆) δ 8.43 (d, J=5.7 Hz, 1H), 8.07 (br s, 1H), 7.66 (br s, 1H), 7.31 (d, J=2.7 Hz, 1H), 7.07 (dd, J=5.7 Hz, 2.7 Hz, 1H), 6.85 (d, J=9.0 Hz, 2H), 6.62 (d, J=8.7 Hz, 2H), 5.17 (broad s, 2H); HPLC EI-MS m/z 230 ((M+H)⁺.

### Step 3: Preparation of N-[4-chloro-3-(trifluoromethyl)phenyl]-N′-{4-[2-carbamoyl-(4-pyridyloxy)]phenyl}urea

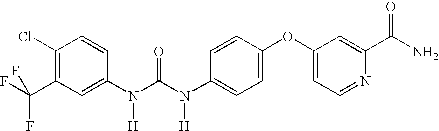

A mixture of 4-chloro-5-trifluoromethylaniline (451 mg, 2.31 mmol, 1.1 equiv.) and 1,1′-carbonyl diimidazole (419 mg, 2.54 mmol, 1.2 equiv.) in anh dichloroethane (5.5 mL) is stirred under argon at 65° C. for 16 h. Once cooled to room temperature, a solution of 4-(4-aminophenoxy)-2-pyridinecarboxamide (480 mg, 2.09 mmol) in anh THF (4.0 mL) is added, and the reaction mixture is stirred at 60° C. for 4 h. The reaction mixture is poured into EtOAc, and the organic layer is washed with water (2×) and a saturated NaCl solution (1×), dried (MgSO₄), filtered, and evaporated in vacuo. Purification using MPLC chromatography (Biotage 0; gradient from 100% EtOAc to 2% MeOH/EtOAc) gave N-[4-chloro-3-(trifluoromethyl)phenyl]-N′-{4-[2-carbamoyl-(4-pyridyloxy)]phenyl}urea as a white solid (770 mg, 82%): TLC (EtOAc) R_{f}=0.11, 100% ethyl acetate ¹H-NMR (DMSO-d₆) δ 9.21 (s, 1H), 8.99 (s, 1H), 8.50 (d, J=5.6 Hz, 1H), 8.11 (s, 1H), 8.10 (s, 1H), 7.69 (broad s, 1H), 7.64 (dd, J=8.2 Hz, 2.1 Hz, 1H), 7.61 (s, 1H), 7.59 (d, J=8.8 Hz, 2H), 7.39 (d, J=2.5 Hz, 1H), 7.15 (d, J=8.9 Hz, 2H), 7.14 (m, 1H); MS LC-MS (MH⁺=451). Anal. calcd for C₂₀H₁₄ClF₃N₄O₃: C, 53.29%; H, 3.13%; N, 12.43%. Found: C, 53.33%; H, 3.21%; N, 12.60%.

Other methods of preparing N-[4-chloro-3-(trifluoromethyl)phenyl]-N′-{4-[2-carbamoyl-(4-pyridyloxy)]phenyl}urea are described in Bankston et al. “A Scaleable Synthesis of BAY 43-9006: A Potent Raf Kinase Inhibitor for the Treatment of Cancer” Org. Proc. Res. Dev. 2002, 6(6), 777-781, and WO 00/42012 and WO 00/41698.

An example of the preparation of sorafenib in the polymorph II is as follows:

903 g of 4-{4-[({[4-chloro-3-(trifluoromethyl)phenyl]amino}carbonyl)amino]phenoxy}-N-methylpyridine-2-carboxamide, prepared as described above, are initially charged in 2700 ml of ethanol. 451.7 g of p-toluenesulfonic acid monohydrate are dissolved in 1340 g of ethanol and added dropwise at room temperature. The suspension is stirred at room temperature for 1 hour, then filtered off with suction, and the residue is washed three times with 830 ml each time of ethanol. The drying is effected at 50° C. under reduced pressure with supply of air. 1129.6 g of the title compound in the polymorph II are obtained.

An example of the preparation of sorafenib in the polymorph I is as follows:

Heating 5 mg of “Sorafenib,”[tosylate salt of 4-{4-[({[4-chloro-3-(trifluoromethyl)phenyl]amino}carbonyl)amino]-phenoxy}-N-methylpyridine-2-carboxamide] in the polymorph II to 200° C. at a heating rate of 20° C./min and subsequently cooling to room temperature at a cooling rate of 2° C./min. The sample is tested thermoanalytically (DSC) and corresponds to the title compound in the polymorph I.

The specific dose level and frequency of dosage may vary, depending upon a variety of factors, including the activity of the active agent, its metabolic stability and length of action, rate of excretion, mode and time of administration, the age, body weight, health condition, gender, diet, baseline hematologic and biologic parameters (e.g., WBCs, granulocytes, platelets, hemoglobin, creatinine, bilirubin, albumin, etc.), etc., of the subject, and the severity, intensity, stage of the cancer, primary site of cancer, size of cancer lesion, presence or extent of metastases, surgical status, disease progression (i.e., aggressive), etc. of the disease.

The compound of the present invention can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, intrathecal, intratumoral, etc. Sorafenib can be administered directly to the site of a tumor, either pre- or post-operatively. It can be administered alone, or in combination with any ingredient(s), active or inactive.

Sorafenib can be administered by the oral route using the pharmaceutical composition of the present invention will generally range, based on body weight, from about 0.01 mg/kg to about 50 mg/kg; from about 1 mg/kg to about 40 mg/kg; from about 5 mg/kg to about 30 mg/kg; from about 10 to about 25 mg/kg; about 10 mg/kg; about 20 mg/kg; about 25 mg/kg; about 30 mg/kg; etc.

Any suitable dosing interval can be used in accordance with the present invention. For example, the compound can be administered once, twice (BID), three, four, etc., times a day. For example, about 100, about 200, about 400 mg, about 500 mg, about 600 mg, or about 800 mg can be administered one, twice, or three times daily.

Sorafenib can be administered at any suitable time. For example, it can be administered routinely as other chemotherapeutic agents; it can be administered as a bolus prior to a surgical intervention; prior to or after radiation, radiofrequency ablation and other energy treatments; post-operatively; pre-operatively; etc.

Sorafenib can be further combined with any other suitable additive or pharmaceutically acceptable carrier. Such additives include any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002).

The compounds can be in any suitable form, without limitation. Forms suitable for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions.

Compounds can be formulated with other ingredients, e.g., “pharmaceutically acceptable carriers” or “excipients” to indicate they are combined with the active drug and can be administered safely to a subject for therapeutic purposes. These include, but are not limited to, antioxidants, preservatives, dyes, tablet-coating compositions, plasticizers, inert carriers, excipients, polymers, coating materials, osmotic barriers, devices and agents which slow or retard solubility, etc.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations.

Non-toxic pharmaceutically acceptable excipients that are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release the drug. Such materials include cocoa butter and polyethylene glycols.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of a compound of Formula I in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of Formula I may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C8-C18 fatty alcohols, saturated or unsaturated C8-C18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

Compositions comprising precursors can also be formulated for controlled release, where release of the active ingredient is regulated or modulated to achieve a desired rate of delivery into the systemic circulation. A controlled release formulation can be pulsed, delayed, extended, slow, steady, immediate, rapid, fast, etc. It can comprise one or more release formulations, e.g. extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of sorafenib.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

From the foregoing description, one skilled in the art can easily ascertain the essential characteristics of this invention and, without departing from the spirit and scope thereof, can make various changes and modifications of the invention to adapt it to various usages and conditions.

TABLE 1
-A-

Adenocarcinoma
Adenocarcinoma Lung Cancer
Adenocystic Carcinoma
Adenoid Cystic Breast Cancer
Adenoid Cystic Carcinoma
Adrenal Gland Cancer:
Adrenal Gland Cancer - Cortical Adenoma
Adrenal Gland Cancer - Cortical Carcinoma
AIDS related disorders:
AIDS-related disorders - Kaposi's sarcoma
AIDS-related disorders, - lymphoma
Advanced Breast Cancer
Aggressive Breast Cancer
Ameloblastoma
Anal (Anus) Cancer, including:
Anal Cancer - Early-Stage (Superficial)
Anal Cancer - Intermediate-Stage I
Anal Cancer - Intermediate-Stage II
Anal Cancer - Intermediate-Stage III
Anal Cancer - Metastatic, Advanced-Stage IV
Anal Cancer - Adenocarcinomas
Anal Cancer - Intraepithelial Neoplasia
Anal Cancer - Bowen's Disease
Anal Cancer - Paget's Disease
Anal Cancer - Small Cell Cancers
Anal Cancer - Squamous Cell Carcinoma
Angiosarcoma
Appendix Cancer (Appendiceal Cancer)
Ascites, Malignant
Astrocytoma

-B-

Barrett's Esophagus
Bartholin Gland Cancer
Basal Cell Carcinoma
Basal Cell Skin Cancer
B-Cell Lymphoma
Bilateral Breast Cancer
Bile Duct Cancer
Bladder Cancer:
Bladder Cancer - Superficial
Bladder Cancer - Muscle-Invasive
Bladder Cancer - Small Cell
Bladder Cancer - Metastases
Bone Cancer:
Bone Cancer - Chordoma
Bone Cancer - Chondrosarcoma
Bone Cancer - Ewing's Sarcoma (Child)
Bone Cancer - Giant Cell
Bone Cancer - Mesenchymal Chondrosarcoma
Bone Cancer - Osteosarcoma
Bone Cancer - Bone Marrow Transplantation
Bone Cancer - Bone Metastases
Bowen's Disease
Borderline Ovarian Cancer
Brain Tumors:
Brain Tumors - Adult
Brain Tumors - Child
Brain Tumors - Astrocytoma
Brain Tumors - Brainstem
Brain Tumors - Cavernous Sinus
Brain Tumors - Chondroma
Brain Tumors - Ependymoma
Brain Tumors - Glioblastoma
Brain Tumors - Glioma
Brain Tumors - Gliomatosis
Brain Tumors - Hemangiopericytoma
Brain Tumors - Medulloblastoma
Brain Tumors - Oligodendroglioma
Brain Tumors - Pineoblastoma/Pinealoma
Brain Tumors - Primary Central Nervous System Lymphoma
Brain Tumors - Thalamic
Brain Tumors - Brainstem Glioma
Breast Cancers Including:
Breast Cancer - Adenoid Cystic
Breast Cancer - Advanced
Breast Cancer - Aggressive
Breast Cancer - Apocrine Carcinoma
Breast Cancer - Bilateral (in both breasts)
Breast Cancer - Comedo Type
Breast Cancer - Ductal Carcinoma In Situ
Breast Cancer - Ductal Carcinoma Invasive
Breast Cancer - Early Stage
Breast Cancer - Estrogen-Negative
Breast Cancer - High-Risk
Breast Cancer - Inflammatory
Breast Cancer - in Pregnancy
Breast Cancer - Lobular, Invasive
Breast Cancer - Lobular Carcinoma In situ (LCIS)
Breast Cancer - Lymphoma
Breast Cancer - Medullary
Breast Cancer - Microinvasive
Breast Cancer - Mucinous
Breast Cancer - Node-Negative
Breast Cancer - Node-Positive
Breast Cancer, Nonepithelial Neoplasms:
Breast Cancer - Nonepithelial Neoplasms - Phyllodes Sarcomas
Breast Cancer - Nonepithelial Neoplasms - Phyllodes Tumors
Breast Cancer - Occult with Lymphatic Metastasis
Breast Cancer - Papillary
Breast Cancer - Postmenopausal
Breast Cancer - Premenopausal
Breast Cancer - Recurrent
Breast Cancer - Sarcoma
Breast Cancer - Secretory (juvenile) Carcinoma
Breast Cancer - Squamous
Breast Cancer - Stage I
Breast Cancer - Stage II
Breast Cancer - Stage III
Breast Cancer - Stage IV
Breast Cancer - Tubular
Breast Cancer - Ulcerating
Breast Cancer with Bladder Metastases
Breast Cancer with Bone Metastases
Breast Cancer with Bone and Lung Metastases
Breast Cancer with Brain Metastases
Breast Cancer with Brain and Liver Metastases
Breast Cancer with Central Nervous System Metastases
Breast Cancer with Liver Metastases
Breast Cancer with Lung Metastases
Breast Cancer with Lymphangitic Metastasis
Breast Cancer with Lymphatic Metastases
Breast Cancer with Lymph Node Metastases
Breast Cancer with Meningeal Metastases
Breast Cancer with Pericardial Effusion
Breast Cancer with Pleura Metastases
Breast Cancer with Pleural Effusion
Breast Cancer with Skin Metastases
Breast Cancer with Spine Metastases
Breast Cancer with Stomach Metastases
Breast Cancer - Breast Reconstruction
Breast Cancer - Comedo Type Breast Cancer
Breast Cancer - Early Stage Breast Cancer
Breast Cancer - Estrogen-Negative Breast Cancer
Breast Cancer - Inflammatory Breast Cancer
Breast Cancer - Invasive Breast Cancer
Breast Cancer - Lobular Breast Cancer
Breast Cancer - Medullary Breast Cancer
Breast Cancer - Metachronous Bilateral
Breast Cancer - Microinvasive Breast Cancer
Breast Cancer - Mucinous Breast Cancer
Breast Cancer - Node-Negative Breast Cancer
Breast Cancer - Node-Positive Breast Cancer
Breast Cancer - Occult Breast Cancer with Lymphatic Metastasis
Breast Cancer - Paget's Disease of the Nipple
Breast Cancer - Papillary Breast Cancer
Breast Cancer - Recurrent Breast Cancer
Breast Cancer - Sarcoma of the Breast
Breast Cancer - Squamous Cell Breast Cancer
Breast Cancer - Synchronous Bilateral Breast Cancer
Breast Cancer - Tubular Breast Cancer
Breast Cancer - Ulcerating Breast Cancer
Bronchial Cancer
Bronchioalveolar Lung Cancer
Buttock Cancer

-C-

Cancer of Unknown Primary Site (CUPS)
Carcinoma of Unknown Primary
Carcinoid of the Appendix
Carcinoid of the Colon
Carcinoid of the Pancreas
Carcinoid of the Rectum
Carcinoid of the Stomach
Carcinoid Syndrome
Carcinoid Tumor
Carcinosarcoma of the Ovary
Carcinosarcoma of the Uterus
Carney's Triad
Cavernous Sinus Cancer
Cecum Cancer
Central Nervous System Lymphoma
Central Nervous System Cancers:
Central Nervous System Cancers - Medulloblastoma
Central Nervous System Cancers - Astrocytomas
Central Nervous System Cancers - Gliomas - Optic, Chiasmal, and Hypothalamic
Central Nervous System Cancers - Cystic Cerebellar Astrocytomas
Central Nervous System Cancers - Diffuse, Fibrillary Astrocytomas - e.g., World Health
Organization (WHO) Grade II
Central Nervous System Cancers - Astrocytoma, e.g., WHO grade III Anaplastic
Astrocytoma, and WHO grade IV
Central Nervous System Cancers - Glioblastoma (GBM)
Central Nervous System Cancers - Pilocytic Astrocytoma
Central Nervous System Cancers - Pleomorphic Xanthoastrocytoma
Central Nervous System Cancers - Subependymal Giant Cell Astrocytomas
Central Nervous System Cancers - Oligodendroglioma - Anaplastic, differentiated
Central Nervous System Cancers - Oligoastrocytoma - Anaplastic, differentiated
Central Nervous System Cancers - Ependymoma - Ellular, Papillary, Clear Cell,
Tanycytic, Myxopapillary
Central Nervous System Cancers - Choroid Plexus Tumor
Central Nervous System Cancers - Meningioma - Atypical and Malignant, Parasagittal,
Falx, Olfactory Groove, Tuberculum Sellae, Sphenoid
Ridge, Cerebellopontine Angle
Central Nervous System Cancers - Schwannoma
Central Nervous System Cancers - Neurofibromas
Central Nervous System Cancers - Hemangioblastomas
Central Nervous System Cancers - Atypical Teratoid/Rhabdoid Tumors
Central Nervous System Cancers - Gorlin's, Turcot, and Li-Fraumeni syndromes
Central Nervous System Cancers - Supratentorial Parenchymal tumors
Central Nervous System Cancers - Medulloepithelioma
Central Nervous System Cancers - Neuroblastoma
Central Nervous System Cancers - Ependymoblastoma
Central Nervous System Cancers - Pineoblastoma
Central Nervous System Cancers - Pineal Parenchymal Tumors
Central Nervous System Cancers - Germinomas
Central Nervous System Cancers - Pituitary Adenoma
Central Nervous System Cancers - Craniopharyngioma
Central Nervous System Cancers - Acoustic Neuromas (Vestibular Schwannomas)
Central Nervous System Cancers - Glomus Jugulare tumors (Paragangliomas)
Central Nervous System Cancers - Chordoma and Chondrosarcoma
Central Nervous System Cancers - Hemangioblastomas
Central Nervous System Cancers - Choroid Plexus Papilloma and Carcinoma
Central Nervous System Cancers - Spinal Axis Tumors
Centrocytic Lymphoma
Cerebral Astrocytoma
Cervix Cancer (Cervical Cancer):
Cervix Cancer (Cervical Cancer), Adenocarcinoma In Situ:
Cervical Cancer, Adenocarcinoma In Situ - Adenoid Basal Carcinoma
Cervical Cancer, Adenocarcinoma In Situ - Adenoma Malignum
Cervical Cancer, Adenocarcinoma In Situ - Adenosquamous Carcinoma
Cervical Cancer, Adenocarcinoma In Situ - Glassy Cell Carcinoma
Cervical Cancer, Adenocarcinoma In Situ - Papillary Serous Carcinoma
Cervix Cancer (Cervical Cancer), Anaplastic Small Cell Carcinoma
Cervix Cancer (Cervical Cancer), Cervical Intraepithelial Neoplasia
Cervix Cancer (Cervical Cancer), Malignant Mixed Müllerian Tumors
Squamous Cell Carcinoma:
Squamous Cell Carcinoma - Large Cell Keratinizing
Squamous Cell Carcinoma - Large Call Nonkeratinizing
Squamous Cell Carcinoma - Papillary
Squamous Cell Carcinoma - Sarcomatoid Squamous Carcinoma
Squamous Cell Carcinoma - Small Cell Carcinoma
Squamous Cell Carcinoma - Verrucous Carcinoma
Chest Tumors (Mediastinum Tumors)
Chiasmal Glioma
Childhood Cancers
Chondroma, Intracranial
Chordoma
Chondrosarcoma:
Chondrosarcoma - Central
Chondrosarcoma - Clear Cell
Chondrosarcoma - Differentiated
Chondrosarcoma - Mesenchymal
Chondrosarcoma - Peripheral
Chorioadenoma Destruens
Choriocarcinoma:
Choriocarcinoma, Syncytiotrophoblast
Choriocarcinoma, Cytotrophoblast
Choroid Plexus Carcinoma
Choroidal Melanoma
Chronic Leukemia
Chronic Lymphocytic Leukemia (CLL)
Chronic Myelogenous Leukemia (CML)
Clear Cell Ovarian Cancer
Clear Cell Sarcoma
CNS Lymphoma
Colon Cancer Including:
Colon Cancer - Amine Precursor Uptake and Decarboxylation Tumor
Colon Cancer - Cecum
Colon Cancer - Lymphoma
Colon Cancer - Lymphoma - B-Cell
Colon Cancer - Lymphoma - T-Cell
Colon Cancer - Hamartomatous Polyposis Syndrome
Colon Cancer - Hereditary Nonpolyposis Colorectal Cancer
Colon Cancer - Mucinous
Colon Cancer, Neuroendocrine Carcinoma:
Colon Cancer - Neuroendocrine Carcinoma - Extrapulmonary Small Cell
Colon Cancer - Neuroendocrine Carcinoma - High-grade
Colon Cancer - Neuroendocrine Carcinoma - Oat Cell
Colon Cancer - Neuroendocrine Carcinoma - Well-differentiated Carcinoid
Colon Cancer - Recurrent
Colon Cancer - Stage A
Colon Cancer - Stage B
Colon Cancer - Stage C
Colon Cancer - Stage D
Colon Cancer - Stage I (Dukes' A)
Colon Cancer - Stage II (Dukes' B)
Colon Cancer - Stage III (Dukes' C)
Colon Cancer - Stage IV
Colon Cancer with Bone Metastases
Colon Cancer with Brain Metastases
Colon Cancer with Buttocks Metastases
Colon Cancer with Liver Metastases
Colon Cancer with Liver and Lung Metastases
Colon Cancer with Lung Metastases
Colon Cancer with Ovarian Metastases
Colon Cancer with Skin Metastases
Colon Cancer - Adenocarcinoma of the Colon - Mucinous (Colloid)
Colon Cancer - Adenocarcinoma of the Colon - Signet Ring
Colon Cancer - Adenomatous Polyps
Colon Cancer - Polyps
Colorectal Cancer, Colon Cancer
Craniopharyngioma
Cutaneous T-Cell Lymphoma

-D-

DCIS - Ductal Carcinoma In Situ
Dermatofibrosarcoma Protuberans
Desmoid Tumor
Desmoplastic Tumor
Diffuse Lymphoma
Diffuse, Large-Cell Lymphoma
Ductal Carcinoma in Situ (DCIS)
Duodenal Cancer
Dysgerminoma

-E-

Early Stage Prostate Cancer
Endochodroma
Endocrine Cancer:
Endocrine Cancer - Multiple Endocrine Neoplasia Type 1 (MEN 1)
Endocrine Cancer - Multiple Endocrine Neoplasia Type 2 (MEN 2)
Endocrine Cancer - MEN 2A
Endocrine Cancer - MEN 2B
Endocrine Cancer - Familial Non-MEN MTC (FMTC)
Endocrine Cancer - Sporadic MTC
Endocrine Cancer - Sporadic Thyroid Neoplasms
Endocrine Cancer - Papillary Thyroid Carcinoma
Endocrine Cancer - Follicular Adenomas
Endometrial Cancer:
Endometrial Cancer - Adenosarcoma
Endometrial Cancer - Epithelial Tumors
Endometrial Cancer - Epithelial Tumors - Clear Cell
Endometrial Cancer - Epithelial Tumors - Endometrioid
Endometrial Cancer - Epithelial Tumors - Mucinous
Endometrial Cancer - Epithelial Tumors - Papillary Endometrioid
Endometrial Cancer - Epithelial Tumors - Papillary Serous
Endometrial Cancer - Epithelial Tumors - Malignant Mixed Müllerian
Endometrial Cancer - Mesenchymal Tumors:
Endometrial Cancer - Mesenchymal Tumors - Endometrial Stromal Sarcoma
Endometrial Cancer - Mesenchymal Tumors - Leiomyosarcoma
Ependymoma
Esophagus Cancer (Esophageal Cancer), Including:
Esophagus Cancer (Esophageal Cancer) - Adenoid Cystic Carcinoma
Esophagus Cancer (Esophageal Cancer) - Mucoepidermoid Carcinoma
Esophagus Cancer of the Gastroesophageal Junction
Esthesioneuroblastoma
Ewing's Sarcoma
Extensive Stage Small Cell Lung Cancer
Extrahepatic Bile Duct Cancer
Extraskeletal Chondrosarcoma
Extraskeletal Ewing's Sarcoma
Extraskeletal Osteosarcoma
Eye Metastases
Eye Cancer
Eye Cancer, Lymphoma

-F-

Fallopian Tube Cancer Including:
Fallopian Tube Cancer - Adenocarcinomas
Fallopian Tube Cancer - Eiomyosarcomas
Fallopian Tube Cancer - Transitional cell carcinomas
Fallopian Tube Cancer - Stage I
Fallopian Tube Cancer - Stage II
Fallopian Tube Cancer - Stage III
Fallopian Tube Cancer - Stage IV
Fallopian Tube Cancer - Carcinoma
Fallopian Tube Cancer - Ovarian Cancer
Familial Adenomatous Polyposis
Fibrosarcoma - See Soft Tissue Sarcomas
Fibrous Histiocytoma
Follicular Lymphoma
Follicular, Small-Cleaved Cell Lymphoma

-G-

Gardner's Syndrome
Gallbladder Cancer, Including:
Gallbladder Cancer - Adenocarcinoma
Gallbladder Cancer - Mucinous Adenocarcinoma
Gallbladder Cancer - Papillary Adenocarcinoma
Gallbladder Cancer - Sarcoma
Gallbladder Cancer - Squamous Cell Carcinoma
Gastric Cancer
Gastrinoma
Gastro-Esophageal (GE Junction) Cancer
Gastrointestinal Lymphoma
Gastrointestinal Stromal Tumors (GST):
Gastrointestinal Stromal Tumors - Autonomic Nerve Tumor
Gastrointestinal Stromal Tumors - Leiomyoblastoma
Gastrointestinal Stromal Tumors - Leiomyosarcoma
Gastrointestinal Stromal Tumors - Pacemaker Call Tumor
Gastrointestinal Stromal Tumors - Plexosarcoma
Neurofibrosarcoma
GE Junction Esophageal Cancer
Germ Cell Tumor:
Germ Cell Tumor - Retroperitoneal Tumor
Germ Cell Tumor - Mediastinum Tumor
Germ Cell Tumor - Undescended Testicular Cancer
Gestational Trophoblastic Disease
Giant Cell Bone Tumor
Glioblastoma
Glioma
Gliomatosis
Gonadal Mesothelioma
Gorlin's Syndrome (Nevoid Basal Cell Carcinoma Syndrome)
Granular Lymphocytic Lymphoma
Granulocytic Leukemia
Granulosa Cell Ovarian Cancer

-H-

Hairy Cell Leukemia
Head and Neck Cancer
Head and Neck Cancer, Adenoid Cystic
Head and Neck Cancer, Squamous Cell
Hemangioendothelioma
Hemangiopericytoma
Hepatocelluar Carcinoma
High-Grade Lymphoma
High-Risk Breast Cancer
Histiocytoma, Malignant
Hodgkin's Disease
Hodgkin's Disease in Pregnancy
Hormone-Refractory Prostate Cancer

-I-

In Situ Breast Cancer
Insulinoma
Intermediate-Grade Lymphoma
Intraocular Melanoma
Invasive Lobular Breast Cancer
Islet Cell Pancreatic Cancer

-J-

Kaposi's Sarcoma
Kidney Cancer (Renal Cell Carcinoma), Including:
Kidney Cancer (Renal Cell Carcinoma) - Birt-Hogg-Dubé (BHD)
Kidney Cancer (Renal Cell Carcinoma) - Chromophobe Renal Carcinoma
Kidney Cancer (Renal Cell Carcinoma) - Clear Cell Renal Carcinoma
Kidney Cancer (Renal Cell Carcinoma) - Hereditary Papillary Renal Cell Carcinoma
(HPRC)
Kidney Cancer (Renal Cell Carcinoma) - Hereditary Leiomyomatosis Renal Cell
Carcinoma (HLRCC)
Kidney Cancer (Renal Cell Carcinoma) - Onocytoma Renal Carcinoma
Kidney Cancer (Renal Cell Carcinoma) - Papillary Renal Carcinoma
Kidney Cancer (Renal Cell Carcinoma) - Papillary Renal Carcinoma, Type 1
Kidney Cancer (Renal Cell Carcinoma) - Papillary Renal Carcinoma, Type 2
Kidney Cancer (Renal Cell Carcinoma) - Von Hippel-Lindau (VHL) Clear Cell Renal
Cell Carcinoma
Kidney Cancer with Lung Metastases
Krukenberg Tumor

-L-

Large-Cell Lung Cancer
Large-Cell Lymphoma
Large-Cell, Diffuse Lymphoma
Larynx Cancer (Laryngeal Cancer)
Leiomyosarcoma:
Leiomyosarcoma - Foot
Leiomyosarcoma - Pulmonary Artery
Leiomyosarcoma - Vena Cava
Leiomyosarcoma - Uterus
Lentigo Maligna
Leukemia:
Leukemia - Acute
Leukemia - Acute Lymphocytic (ALL)
Leukemia - Acute Megakaryoblastic leukemia
Leukemia - Acute Monocytic (AML)
Leukemia - Acute Myelogenous (AML)
Leukemia - Acute Myeloid (AML)
Leukemia - Acute Promyelocytic (APL)
Leukemia - Chronic
Leukemia - B-Cell Chronic Lymphocytic Leukemia
Leukemia - Chronic granulocytic leukemia
Leukemia - Chronic Lymphocytic (CLL)
Leukemia - Chronic Myelogenous
Leukemia - Chronic Myeloid (CML)
Leukemia - Chronic Myelomonocytic (CMML)
Leukemia - CML, Philadelphia-Negative
Leukemia - CMML
Leukemia - Cutis
Leukemia - Granulocytic
Leukemia - Granulocytic Sarcomas and Chloromas (Leukemic tumors)
Leukemia - Hairy Cell
Leukemia - Infant Acute Megakaryoblastic Leukemia
Leukemia - Large Granular Lymphocyte Leukemia
Leukemia - Lymphoblastic
Leukemia - Mature B-Cell Acute Lymphocytic Leukemia
Leukemia - Precursor B-Lymphoblastic Leukemia
Leukemia - Philadelphia Chromosome-Positive Acute Lymphocytic Leukemia
Leukemia - Prolymphocytic
Leukemia - Promyelocytic
Leukemia - Secondary Myeloid Leukemia
Leukemia - T-Cel
Li-Fraumeni syndrome
Linitis Plastica
Limited Stage Small Cell Lung Cancer
Lip Cancer
Liposarcoma
Liver Cancer Including:
Liver Cancer - Angiosarcoma
Liver Cancer - Cholangiocarcinoma (Bile Duct Cancer):
Liver Cancer - Cholangiocarcinoma (Bile Duct Cancer) - Distal
Liver Cancer - Cholangiocarcinoma (Bile Duct Cancer) - Hilar (Klatskin's
Tumors)
Liver Cancer - Cholangiocarcinoma (Bile Duct Cancer) - Intrahepatic
Liver Cancer - Cholangiocellular Carcinoma
Liver Cancer - Emangioendotheliomas
Liver Cancer - Epithelioid Hemangioendothelioma
Liver Cancer - Fibrosarcoma
Liver Cancer - Leiomyosarcoma
Liver Cancer - Hemangioendothelioma
Liver Cancer - Hemangiosarcoma
Liver Cancer - Hepatic Cystadenocarcinoma
Liver Cancer - Hepatoblastoma
Liver Cancer - Hepatocellular Carcinoma (Liver Cell Carcinoma):
Liver Cancer - Hepatocellular Carcinoma (Liver Cell Carcinoma) -
Carcinosarcoma
Liver Cancer - Hepatocellular Carcinoma (Liver Cell Carcinoma) - Childhood
Liver Cancer - Hepatocellular Carcinoma (Liver Cell Carcinoma) - Clear Cell
Liver Cancer - Hepatocellular Carcinoma (Liver Cell Carcinoma) - Combined
Liver Cancer - Hepatocellular Carcinoma (Liver Cell Carcinoma) -
Fibrolamellar
Liver Cancer - Hepatocellular Carcinoma (Liver Cell Carcinoma) - Giant Cell
Liver Cancer - Hepatocellular Carcinoma (Liver Cell Carcinoma) - Sclerosing,
Hepatoblastoma
Liver Cancer - Hepatocellular Carcinoma (Liver Cell Carcinoma) - Spindle Cell
Liver Cancer - Histiocytoma
Liver Cancer - Lymphoma
Liver Cancer - Malignant Fibrous
Liver Cancer - Malignant Schwannoma
Liver Cancer - Mesenchymal sarcoma
Liver Cancer - Metastases
Liver Cancer - Mixed Hepatocellular Cholangiocarcinoma
Liver Cancer - Osteosacoma
Liver Cancer - Rhabdomyosarcoma
Liver Cancer - Stage I
Liver Cancer - Stage II
Liver Cancer - Stage III
Liver Cancer - Stage IV
Liver Cancer - Recurrent Liver Cancer
Liver Cancer - Undifferentiated Liver Cancer
Lobular Carcinoma in Situ (LCIS)
Lobular Hyperplasia
Low-Grade Lymphoma
Lung Cancer:
Lung Cancer - Adenocarcinoma
Lung Cancer - Bronchioalveolar
Lung Cancer - Cystic Mucinous
Lung Cancer - Extrapulmonary Small Cell Anaplastic Carcinoma - Limited
Lung Cancer - Extrapulmonary Small Cell Anaplastic Carcinoma - Extensive
Lung Cancer - Germ Cell
Lung Cancer - Giant Cell
Lung Cancer - Large-Cell
Lung Cancer - Mucinous
Lung Cancer - Non-Small Cell Lung Cancer - Including:
Lung Cancer - Non-Small Cell Lung Cancer - (NSCLC)
Lung Cancer - Non-Small Cell Lung Cancer with Bone Metastases
Lung Cancer - Non-Small Cell Lung Cancer with Brain Metastases
Lung Cancer - Non-Small Cell Lung Cancer with Liver Metastases
Lung Cancer - Non-Small Cell Lung Cancer with Lymph Node Metastases
Lung Cancer - Non-Small Cell Lung Cancer with Skin Metastases
Lung Cancer - Non-Small Cell Lung Cancer - Stage I
Lung Cancer - Non-Small Cell Lung Cancer - Stage IIA
Lung Cancer - Non-Small Cell Lung Cancer - Stage IIB
Lung Cancer - Non-Small Cell Lung Cancer - Stage IIIA
Lung Cancer - Non-Small Cell Lung Cancer - Stage IIIB
Lung Cancer - Non-Small Cell Lung Cancer - Stage IV
Lung Cancer - Non-Small Cell Lung Cancer - Recurrent
Lung Cancer - Adenocarcinomas:
Lung Cancer - Adenocarcinomas - Acinar
Lung Cancer - Adenocarcinomas, Bronchioloalveolar Carcinoma
Lung Cancer - Adenocarcinomas, Bronchioloalveolar Carcinoma,
Nonmucinous (Clara Cell/type II Pneumocyte Type)
Lung Cancer - Adenocarcinomas, Bronchioloalveolar Carcinoma, Mixed
Mucinous and Nonmucinous/Indeterminate cell type
Lung Cancer - Adenocarcinomas, Bronchioloalveolar Carcinoma,
Mucinous (Goblet Cell) type
Lung Cancer - Adenocarcinomas - Clear Cell Adenocarcinoma
Lung Cancer - Adenocarcinomas - Mucinous (Colloid) Adenocarcinoma
Lung Cancer - Adenocarcinomas - Mucinous Cystadenocarcinoma
Lung Cancer - Adenocarcinomas - Papillary
Lung Cancer - Adenocarcinomas - Signet-Ring Adenocarcinoma
Adenocarcinomas - Solid Adenocarcinoma with Mucin Formation
Lung Cancer - Adenocarcinomas - Well-differentiated Fetal Adenocarcinoma
Adenosquamous Carcinoma
Atypical Adenomatous Hyperplasia
Carcinoid Tumors, Atypical
Carcinoid Tumors, Typical
Carcinosarcoma
Diffuse Idiopathic Pulmonary Neuroendocrine Cell Hyperplasia
Epidermoid Carcinoma
Giant Cell Carcinoma
Large Cell Carcinoma:
Large Cell Carcinoma - Basaloid Carcinoma
Large Cell Carcinoma - Clear Cell Carcinoma
Large Cell Carcinoma - Combined Large Cell Neuroendocrine Carcinoma
Large Cell Carcinoma - Large Cell Carcinoma with Rhabdoid Phenotype
Large Cell Carcinoma - Large Cell Neuroendocrine Carcinoma
Large Cell Carcinoma - Lymphoepithelioma-like Carcinoma
Pleomorphic Carcinoma
Pulmonary Blastoma
Spindle Cell Carcinoma
Squamous Cell Carcinomas:
Squamous Cell Carcinomas - Basaloid
Squamous Cell Carcinomas - Clear Cell
Squamous Cell Carcinomas - Papillary
Squamous Cell Carcinomas - Small Cell
Lung Cancer, Squamous Dysplasia/Carcinoma in situ
Lung Cancer, Superior Sulcus Tumors (Pancoast tumors)
Lung Cancer, Unclassified
Lung Cancer, Recurrent
Lung Cancer, Small Cell:
Lung Cancer - Small Cell - Combined small cell carcinoma
Lung Cancer - Small Cell - Mixed small cell/large cell carcinoma
Lung Cancer - Small Cell - Pure small cell
Lung Cancer - Small Cell - Extensive Stage
Lung Cancer - Small Cell - Limited Stage
Lung Cancer - Small Cell with Brain Metastases
Lung Cancer, Squamous
Lung Cancer, Stage I
Lung Cancer, Stage II
Lung Cancer, Stage III
Lung Cancer, Stage IIIA
Lung Cancer, Stage IIIB
Lung Cancer, Stage IV
Lung Cancer with Adrenal Gland Metastases
Lung Cancer with Bone Metastases
Lung Cancer with Brain Metastases
Lung Cancer with Liver Metastases
Lung Cancer with Lymph Node Metastases
Lung Cancer with Ovarian Metastases
Lung Cancer with Skin Metastases
Lung Metastases
Lymph Node Excision
Lymphangitic Metastasis
Lymph Node Metastases
Lymphoblastic Leukemia
Lymphoblastic Lymphoma
Lymphocytic Lymphoma
Lymphoma:
Lymphoma - Lymphoblastic Lymphoma
Lymphoma - Lymphocytic Lymphoma
Lymphoma - Lymphocytic, Large Granular
Lymphoma - Lymphocytic, Small
Lymphoma - AIDS Related
Lymphoma - Anaplastic
Lymphoma - Anaplastic Large Cell (ALCL)
Lymphoma - Breast
Lymphoma - B-Cell
Lymphoma - B-Cell Large- Lymphomatoid Granulomatosis Type
Lymphoma - B-Cell Large Diffuse
Lymphoma - B-Cell Monocytoid
Lymphoma - Central Nervous System
Lymphoma - Cutaneous T-Cell:
Lymphoma - Cutaneous T-Cell - Mycosis Fungoides, Agetoid Reticulosis or
Woringer-Kolopp disease
Lymphoma, Granulomatous slack skin:
Lymphoma - Granulomatous slack skin - Lymphomatoid
Lymphoma - Granulomatous slack skin - Papulosis
Lymphoma - Granulomatous slack skin - Primary
Lymphoma - Granulomatous slack skin - Cutaneous
Lymphoma - CD30+ Large T-cell Lymphoma
Lymphoma - CD56+ “Nasal-Type” NK/T-Cell Lymphoma
Lymphoma - Diffuse
Lymphoma - Diffuse, Large-Cell
Lymphoma - Enteropathy-Type T-Cell Lymphoma
Lymphoma - Eye
Lymphoma - Follicular
Lymphoma - Follicular, Small-Cleaved Cell
Lymphoma - Gastrointestinal
Lymphoma - Granular, Lymphocytic
Lymphoma - Hepatosplenic T-Cell Lymphoma
Lymphoma - High-Grade
Lymphoma - Hodgkin's Disease:
Lymphoma - Hodgkin's Disease - Paragranuloma
Lymphoma - Hodgkin's Disease - Granuloma
Lymphoma - Hodgkin's Disease - Sarcoma
Lymphoma - Hodgkin's Disease - Nodular Lymphocyte-Predominant Hodgkin's
Lymphoma
Lymphoma - Hodgkin's Disease - Nodular Lymphocyte-Predominant Hodgkin's
Lymphoma
Lymphoma - Hodgkin's disease - Mixed-Cellularity Hodgkin's Lymphoma
Lymphoma - Hodgkin's disease - Nodular Sclerosis Hodgkin's Lymphoma
Lymphoma - Hodgkin's disease - Lymphocyte-Depleted Hodgkin's Lymphoma
Lymphoma - Hodgkin's disease - Lymphocyte-Predominant Hodgkin's
Lymphoma
Lymphoma - Hodgkin's disease - Lymphocyte-Rich Classic Hodgkin's
Lymphoma
Lymphoma - Intermediate Grade
Lymphoma - Hodgkin's disease, Intravascular Large B-Cell Lymphoma
Lymphoma - Large-Cell
Lymphoma - Large-Cell, Diffuse
Lymphoma - Low-Grade
Lymphoma - Hodgkin's disease, Low-Grade B-Cell Lymphoma of Mucosa-
Associated Lymphoid Tissue
Lymphoma - Hodgkin's disease, Lymphoplasmacytic lymphoma
Lymphoma - MALT
Lymphoma - Mantle Cell
Lymphoma - Non-Hodgkin's, Including:
Lymphoma - Non-Hodgkin's - High Grade
Lymphoma - Non-Hodgkin's - Intermediate Grade
Lymphoma - Non-Hodgkin's - Low Grade
Lymphoma - Non-Hodgkin's - Burkitt's & Atypical Burkitt's
Lymphoma - Non-Hodgkin's - Diffuse
Lymphoma - Non-Hodgkin's - Non-Burkitt's
Lymphoma - Non-Hodgkin's - T-Cell, Cutaneous
Lymphoma - Non-Hodgkin's - Immunoblastic Diffuse
Lymphoma - Non-Hodgkin's - Large Cell Follicular
Lymphoma - Non-Hodgkin's - Mixed Cell Diffuse
Lymphoma - Non-Hodgkin's - Small Cleaved Cell Follicular
Lymphoma - Non-Hodgkin's - Small Lymphocytic
Lymphoma - Non-Hodgkin's - Mixed Follicular
Lymphoma - Non-Hodgkin's - Small Cleaved Cell Diffuse,
Lymphoma - Non-Hodgkin's - Intermediately Differentiated Diffuse
Lymphoma - Non-Hodgkin's - Cutaneous T-cell
Lymphoma, Orbital
Lymphoma, Peripheral T-Cell
Precursor B-Lymphoblastic Lymphoma
Lymphoma, Primary Central Nervous System
Primary Effusion Lymphoma
Primary leptomeningeal lymphoma
Primary Mediastinal (Thymic) Large B-Cell LymphomaPrimary spinal cord
lymphoma
Richter's lymphoma
Lymphoma, Small Cleaved Cell
Lymphoma, Soft tissue
Splenic Marginal Zone Lymphoma with or without Villous Lymphocytes
Subcutaneous Panniculitis-Like T-Cell Lymphoma
Lymphoma, T-Cell
Lymphoma, T-Cell, Cutaneous
T-Cell-Rich/Histiocyte-Rich Large B-Cell Lymphoma
Lymphoma, Thyroid

-M-

Malignant Ascites
Malignant Carcinoid Syndrome:
Malignant Carcinoid Syndrome - Atypical
Malignant Carcinoid Syndrome - Typical
Malignant Hemangiopericytoma
Malignant Fibrous Histiocytoma
Malignant Peripheral Nerve Sheath Tumor
Malignant Pleural Effusion
Malignant Pleural Mesothelioma
Malignant Schwannoma
MALT Lymphoma
Mantle Cell Lymphoma
Mediastinal Neoplasms, including:
Mediastinal Neoplasms - Chemodectoma (Paraganglioma)
Mediastinal Neoplasms - Choriocarcinoma
Mediastinal Neoplasms - Endodermal Sinus Tumors (Yolk Sac Tumors)
Mediastinal Neoplasms - Fibroelastoma
Mediastinal Neoplasms - Fibroma
Mediastinal Neoplasms - Fibrosarcoma
Mediastinal Neoplasms - Ganglioneuroblastoma
Mediastinal Neoplasms - Ganglioneuroma
Mediastinal Neoplasms - Granular Cell Myoblastomas
Mediastinal Neoplasms - Hemangioendothelioma
Mediastinal Neoplasms - Hemangioma
Mediastinal Neoplasms, Histiocytic Lymphoma
Mediastinal Neoplasms, Immature Teratomas
Mediastinal Neoplasms, Leiomyoma
Mediastinal Neoplasms, Leiomyosarcoma
Mediastinal Neoplasms, Lipoma
Mediastinal Neoplasms, Liposarcoma
Mediastinal Neoplasms, Lymphangioma
Mediastinal Neoplasms, Lymphangiomyoma
Mediastinal Neoplasms, Malignant Fibrous Histiocytoma
Mediastinal Neoplasms, Mesenchymoma
Mediastinal Neoplasms, Mixed Embryonal Cell Carcinoma,
Mediastinal Neoplasms, Mixed Embryonal Cell Carcinoma, with Endodermal
Sinus Elements
Mediastinal Neoplasms, Mixed Embryonal Cell Carcinoma, with Seminomatous
Elements
Mediastinal Neoplasms, Mixed Embryonal Cell Carcinoma, with Tetroid
Elements
Mediastinal Neoplasms, Mixed Embryonal Cell Carcinoma, with Trophoblastic
Elements
Mediastinal Neoplasms, Myxoma
Mediastinal Neoplasms, Neuroblastoma
Mediastinal Neoplasms, Neurofibroma
Mediastinal Neoplasms, Neuroilemoma (schwannoma)
Mediastinal Neoplasms, Neurosarcoma
Mediastinal Neoplasms, Nonseminomatous
Mediastinal Neoplasms, Permeative Neuroectodermal Tumor
Mediastinal Neoplasms, Pheochromocytoma
Mediastinal Neoplasms, Pure Embryonal Cell Carcinoma
Mediastinal Neoplasms, Rhabdomyosarcoma
Mediastinal Neoplasms, Seminoma
Mediastinal Neoplasms, Xanthogranuloma
Medulary Thyroid Carcinoma
Medulloblastoma
Melanomas including:
Melanomas, Amelanotic melanoma
Melanomas, Cutaneous
Melanomas, Intraocular
Melanomas, Uveal-spindle cell (A, B, or both), mixed cell, epithelioid cell
Melanomas, Soft Tissue
Melanomas, Extraocular Extension
Melanomas, Multiple Choroidal Malignant Melanoma
Melanomas, with Brain Metastases
Melanomas, with Liver Metastases
Melanomas, with Lung Metastases
Melanomas, Melanoma-associated retinopathy
Meningeal Cancer
Meningeal Metastases
Meningioma
Merkel Cell Carcinoma
Merkel Cell Skin Cancer
Mesothelioma:
Mesothelioma, Biphasic
Mesothelioma, Epithelial
Mesothelioma, Poorly Differentiated
Mesothelioma, Sarcomatoid
Mesenchymal Chondrosarcoma
Metastatic carcinoma of unknown primary site:
Metastatic carcinoma of unknown primary site, poorly differentiated neoplasm
Metastatic carcinoma of unknown primary site, poorly differentiated carcinoma (with or
without features of adenocarcinoma
Metastatic carcinoma of unknown primary site, well-differentiated and moderately well-
differentiated adenocarcinoma
Metastatic carcinoma of unknown primary site, Metastatic gestational Choriocarcinoma
Metastatic Melanoma
Mixed Mullerian Tumor
Mucinous Carcinoma
Multiple Myeloma
Muscle-Invasive Bladder Tumor
Mycosis Fungoides
Myeloblastomas (chloromas or granulocytic sarcomas)
Myelodysplasia
Myelodysplastic Syndrome
Myeloma, Bence-Jones
Myeloproliferative Disorder
Myelodysplastic syndromes (MDS):
Myelodysplastic syndromes (MDS), refractory anemia (RA),
Myelodysplastic syndromes (MDS), preleukemia, or smoldering leukemia
Myelodysplastic syndromes (MDS), hypocellular MDS
Myelodysplastic syndromes (MDS), with extensive fibrosis
Myelodysplastic syndromes (MDS), unclassifiable MDS
Myelodysplastic syndromes (MDS), 5q-syndrome
Myelodysplastic syndromes (MDS), with Myelofibrosis
Myelodysplastic Syndromes (MDS), hypoplastic
Myelodysplastic Syndromes (MDS), with Eosinophilia or Basophilia

-N-

Nail/Nail Bed Cancer
Nasal Cavity Cancer
Nasopharynx Cancer
Neck, Radical Dissection
Neoplastic Meningitis
Nerve Sheath Tumors
Neuroblastoma (Adult)
Neuroblastoma (Child)
Neuroendocrine Tumors:
Neuroendocrine Tumors, Carcinoids (Foregut, Hindgut, Midgut)
Neuroendocrine Tumors, Glandular
Neuroendocrine Tumors, Insular
Neuroendocrine Tumors, Mixed
Neuroendocrine Tumors, Trabecular
Neuroendocrine Tumors, Undifferentiated
Neurofibromatosis Type I (Von Reckling-Hausen's Disease)
Neurologic Paraneoplastic Syndrome
Nose Tumor

-O-

Ocular Melanoma
Olfactory Cancer
Oligodendroglioma
Optic Glioma
Oral Cancer
Orbital Lymphoma
Oropharynx Cancer
Osteochondroma
Osteogenic Sarcoma
Osteoid Tumors
Osteosarcoma:
Osteosarcoma, Central Medullary
Osteosarcoma, Low-Grade Intraosseous
Osteosarcoma, Paget's Sarcoma
Osteosarcoma, Parosteal
Osteosarcoma, Periosteal
Osteosarcoma, Small Cell
Ovarian Cancer (Adult)
Ovarian Cancer (Child)
Ovarian Cancer, Advanced
Ovarian Cancer, Androblastoma:
Ovarian Cancer, Androblastoma, Gynandroblastoma
Ovarian Cancer, Androblastoma, Hilus Cell Tumor
Ovarian Cancer, Androblastoma, Sertolid Cell Tumor (Tubular Adenoma of Pick)
Ovarian Cancer, Androblastoma, Sertoli-Leydig Cell Tumor
Ovarian Cancer, Androblastoma, Leydig Cell Tumor
Ovarian Cancer, Androblastoma, Tubular Androblastoma
Ovarian Cancer, Androblastoma, Unclassified
Ovarian Cancer, Borderline:
Ovarian Cancer, Borderline, Endometrioid
Ovarian Cancer, Borderline, Mucinous,
Ovarian Cancer, Borderline, Mucinous, Micropapillary Serous Carcinoma
Ovarian Cancer, Borderline, Mucinous, Pseudomyxoma Peritonei Serous
Ovarian Cancer, Carcinosarcoma
Ovarian Cancer, Endometrioid Tumor:
Ovarian Cancer, Endometrioid Tumor, Brenner Tumor
Ovarian Cancer, Endometrioid Tumor, Carcinoma
Ovarian Cancer, Endometrioid Tumor, Carcinoma, Adenoacanthoma
Ovarian Cancer, Endometrioid Tumor, Carcinoma, Adenocarcinoma
Ovarian Cancer, Endometrioid Tumor, Carcinoma, Malignant Adenofibroma,
Ovarian Cancer, Endometrioid Tumor, Carcinoma, Malignant
Adenofibroma,
Ovarian Cancer, Endometrioid Tumor, Carcinoma, Cystadenofibroma
Ovarian Cancer, Endometrioid Tumor, Clear Cell (mesonephroid) Tumor,
Ovarian Cancer, Endometrioid Tumor, Clear Cell (mesonephroid) Tumor,
Adenocarcinoma
Ovarian Cancer, Endometrioid Tumor, Clear Cell (mesonephroid) Tumor,
Carcinoma
Ovarian Cancer, Endometrioid Tumor, Endometrioid Stromal Sarcoma
Ovarian Cancer, Endometrioid Tumor, Mesodermal (Mullerian) Mixed Tumor,
Ovarian Cancer, Endometrioid Tumor, Mesodermal (Mullerian) Mixed Tumor,
Heterologous
Ovarian Cancer, Endometrioid Tumor, Mesodermal (Mullerian) Mixed Tumor,
Homologous
Ovarian Cancer, Endometrioid Tumor, Mixed Epithelial Tumor
Ovarian Cancer, Endometrioid Tumor, Unclassified
Ovarian Cancer, Endometrioid Tumor, Undifferentiated Carcinoma
Ovarian Cancer, Epithelial
Ovarian Cancer, Germ Cell:
Ovarian Cancer, Germ Cell, Dysgerminoma
Ovarian Cancer, Germ Cell, Embryonal Carcinoma
Ovarian Cancer, Germ Cell, Endodermal Sinus Tumor
Ovarian Cancer, Germ Cell, Polyembryoma
Ovarian Cancer, Gonadoblastoma:
Ovarian Cancer, Gonadoblastoma, Mixed
Ovarian Cancer, Gonadoblastoma, Pure
Ovarian Cancer, Granulosa-Stromal Cell Tumor:
Ovarian Cancer, Granulosa-Stromal Cell Tumor, Fibroma
Ovarian Cancer, Granulosa-Stromal Cell Tumor, Granulosa Cell Tumor
Ovarian Cancer, Granulosa-Stromal Cell Tumor, Thecoma-Fibroma Tumor
Ovarian Cancer, Granulosa-Stromal Cell Tumor, Unclassified
Ovarian Cancer, Lipoid Cell Tumor:
Ovarian Cancer, Lipoid Cell Tumor, Choriocarcinoma
Ovarian Cancer, Lipoid Cell Tumor, Mixed
Ovarian Cancer, Lipoid Cell Tumor, Teratoma,
Ovarian Cancer, Lipoid Cell Tumor, Teratoma, Carcinoid
Ovarian Cancer, Lipoid Cell Tumor, Teratoma, Immature
Ovarian Cancer, Lipoid Cell Tumor, Teratoma, Mature Dermoid Cyst with
Malignant Transformation
Ovarian Cancer, Lipoid Cell Tumor, Teratoma, Monodermal
Ovarian Cancer, Lipoid Cell Tumor, Teratoma, Struma Ovarii
Ovarian Cancer, Lipoid Cell Tumor, Teratoma, Struma Ovarii and Carcinoid
Ovarian Cancer, Malignant Mucinous Tumor:
Ovarian Cancer, Malignant Mucinous Tumor, Adenocarcinoma
Ovarian Cancer, Malignant Mucinous Tumor, Adenocarcinoma
Cystadenocarcinoma
Ovarian Cancer, Malignant Mucinous Tumor, Malignant Adenofibroma
Ovarian Cancer, Malignant Mucinous Tumor, Malignant Adenofibroma
Cystadenofibroma
Ovarian Cancer, Malignant Serous Tumor:
Ovarian Cancer, Malignant Serous Tumor, Adenocarcinoma,
Ovarian Cancer, Malignant Serous Tumor, Adenocarcinoma, Papillary
Adenocarcinoma
Ovarian Cancer, Malignant Serous Tumor, Adenocarcinoma, Papillary
Cystadenocarcinoma
Ovarian Cancer, Malignant Serous Tumor, Malignant Adenofibroma,
Ovarian Cancer, Malignant Serous Tumor, Malignant Adenofibroma,
Cystadenofibroma
Ovarian Cancer, Malignant Serous Tumor, Surface Papillary Carcinoma
Ovarian Cancer, Peritoneal
Ovarian Cancer, Recurrent
Ovarian Cancer, Sarcoma
Ovarian Cancer, Stage I
Ovarian Cancer, Stage II
Ovarian Cancer, Stage III
Ovarian Cancer, Stage IV
Ovarian Cancer, Stromal Cell
Ovarian Cancer with Bone Metastases
Ovarian Cancer with Liver Metastases
Ovarian Cancer with Lung Metastases
Ovarian Cancer with Meningeal Metastases
Ovarian Cancer with Pleural Effusion
Ovarian Cancer with Spleen Metastases

-P-

Paget's Disease of the Breast
Pancreatic Cancer including:
Pancreatic Cancer, Acinar cell carcinoma
Pancreatic Cancer, Adenocarcinoma:
Pancreatic Cancer - Adenocarcinoma, Anaplastic
Pancreatic Cancer - Adenocarcinoma, Adenosquamous
Pancreatic Cancer - Adenocarcinoma, Colloid Ductal (mucinous
noncystics)
Pancreatic Cancer - Adenocarcinoma, Ductal Adenocarcinoma
Pancreatic Cancer - Adenocarcinoma, Medullary
Pancreatic Cancer - Adenocarcinoma, Signet-ring Cell
Pancreatic Cancer - Adenocarcinoma, Undifferentiated carcinoma with
osteoclast-like giant cells
Pancreatic Cancer- Carcinoid
Pancreatic Cancer, Cystic Neoplasms:
Pancreatic Cancer, Cystic Neoplasms, Intraductal Papillary Mucinous
Neoplasms
Pancreatic Cancer, Cystic Neoplasms, Mucinous Cystic Neoplasms
Pancreatic Cancer, Cystic Neoplasms, Mucinous Cystic Neoplasms Borderline
Pancreatic Cancer, Cystic Neoplasms, Mucinous Cystic Neoplasms
Cystadenocarcinoma
Pancreatic Cancer, Cystic Neoplasms, Mucinous Cystic Neoplasms
Cystadenoma
Pancreatic Cancer, Cystic Neoplasms, Mucinous Cystic Neoplasms Mucinous
Cystic Neoplasm with In Situ Carcinoma
Pancreatic Cancer, Cystic Neoplasms, Serous Cyctic Neoplasms
Pancreatic Cancer, Inoperable
Pancreatic Cancer, Islet Cell
Pancreatic Cancer, Islet Cell, gastrinoma
Pancreatic Cancer, Islet Cell, insulinoma
Pancreatic Cancer, Islet Cell, lucagonoma
Pancreatic Cancer - Operable
Pancreatic Cancer - Pancreatoblastoma
Pancreatic Cancer with Liver Metastases
Pancreatic Cancer - Stage I
Pancreatic Cancer - Stage II
Pancreatic Cancer - Stage III
Pancreatic Cancer - Stage IVA
Pancreatic Cancer - Stage IVB
Pancreatic Cancer - Pancreatic endocrine tumors (PET)
Pancreatic Cancer - PPoma
Pancreatic Cancer - VIPoma
Pancreatic Cancer, Recurrent Pancreactic Cancer
Pancreatic Cancer, Islet cell tumors, gastrinoma
Pancreatic Cancer, Borderline Pancreatic Tumors
Paraganglioma
Paranasal Sinus Cancer
Paraneoplastic Syndromes
Ectopic Adrenocorticotropic Hormone Syndrome:
Syndrome of Inappropriate Antidiuretic Hormone Production
Protein-Losing Enteropathy
Cutaneous paraneoplastic syndrom
Paget's disease of the breast Pancreatic Cancer, Islet Cell
Acrokeratosis paraneoplastica
Melanosis
acanthosis nigricans
Sweet's syndrome
Necrolytic migratory erythema
Multicentric reticulohistiocytosis
Paraneoplastic pemphigus
Acquired ichthyosis
Dermatomyositis
Pachydermoperiostosis
Hypertrichosis lanuginosa acquisita (malignant down)
Muir-Torre syndrome
Subacute Sensory Neuronopathy and Encephalomyeloneuritis
Limbic Encephalitis
Acute polyradiculoneuropathy
Hypertrophic Osteoartbropathy
Dermatomyositis
Paraneoplastic Peripheral Neuropathies
Paraneoplastic Motor Neuron Disorders
Paraneoplastic Vision Loss
Neuromuscular Junction Disorders
Paraneoplastic neuromyotonia
paraneoplastic encephalomyelitis
Paraneoplastic parkinsonian syndromes
Parathyroid neoplasia
Parathyroid carcinoma
Parotid Gland Cancer
Pelvic Tumor
Penis Cancer:
Penis Cancer - Advanced Penile Cancer
Penis Cancer - Balantitis Xerotica Obliterans
Penis Cancer - Buschke-Löwenstein Tumor
Penis Cancer - Carcinoma In Situ
Penis Cancer - Invasive Penile Cancer
Penis Cancer - Penile Verrucous Carcinoma
Periampullary Cancer
Pericardial Effusion:
Pericardial Mesothelioma
Peripheral Nerve Sheath Tumor
Peripheral T-Cell Lymphoma
Peritoneal cancer:
Disseminated peritoneal adenomucinosis
Extraovarian Serous Carcinoma
Extraovarian Peritoneal Serous Papillary Carcinoma
Serous Surface Carcinoma of the Peritoneum
Serous Surface Papillary Carcinoma
Papillary Serous Carcinoma of the Peritoneum
Multiple Focal Peritoneal Papillary Carcinoma
Primary peritoneal carcinoma
Primary Peritoneal Mesothelioma:
Adenomatoid
localized fibrous mesothelioma
multicystic and well-differentiated papillary peritoneal mesotheliomas
epithelial
sarcomatoid
mixed (elements of both epithelial and sarcomatoid
Primary Peritoneal Papillary Serous Adenocarcinoma
Peritoneal Carcinomatosis:
mucinous adenocarcinoma of the appendix and colon
adenocarcinoma of the colon, stomach, or pancreas
Pseudomyxoma Peritonei
Peutz-Jeghers syndrome
Pheochromocytoma
Pinealoma
Pineoblastoma
Placental-Site Trophoblastic Tumors
Plasmacytoma
Plasma Cell Neoplasms:
plasma cell myeloma
monoclonal gammopathy of unknown significance (MGUS)
astleman's disease
α-heavy-chain disease
Waldenström's macroglobulinemia (WM)
plasma cell leukemia
multiple myeloma (MM)
Solitary Plasmacytoma
Indolent MyelomaPleura Cancer
Pleura Metastases
Pleural Effusion, Malignant
Pleural Mesothelioma, Malignant
Preleukemia
Primary Central Nervous System Lymphoma
Primitive Neuroectodermal Tumor (PNET) (Adult)
Primitive Neuroectodermal Tumor (PNET) (Child)
Prolymphocytic Leukemia
Promyelocytic Leukemia
Prophylactic Mastectomy
Prostate Cancer:
Acinar Adenocarcinoma
Ductal Carcinoma
Intralobular Acinar Carcinoma
Malignant Mesenchymal Tmors
Angiosarcoma
Chondrosarcoma
Liposarcoma
Myosarcoma
Osteosarcoma
Rhabdomyosarcomas
Leiomyosarcomas
Mucinous Carcinoma
Proximal Ductal Epithelium Adenocarcinoma
Scirhous Patern Tumors
Transitional Cell Tumors
Prostate Cancer, Early-Stage
Prostate Cancer, Hormone Refractory
Prostate Cancer in the Elderly
Prostate Cancer, Small Cell
Prostate Cancer, Stage A (Stage I)
Prostate Cancer, Stage B (Stage II)
Prostate Cancer, Stage C (Stage III)
Prostate Cancer, Stage D (Stage IV)
Prostate Cancer, Transitional Cell
Prostate Cancer with Bone Metastases
Prostatic Intraepithelial Neoplasia (PIN)
PSA
Pseudomyxoma Peritonei

-R-

Rectal Cancer, Stage I
Rectal Cancer, Stage II
Rectal Cancer, Stage III
Rectal Cancer with Brain Metastases
Rectal Cancer with Liver Metastases
Recurrent Colon Cancer
Recurrent Colorectal Cancer
Recurrent Lung Cancer
Recurrent Melanoma
Recurrent Ovarian Cancer
Recurrent Prostate Cancer
Recurrent Rectal Cancer
Relapsed Lymphoma
Renal Cell Carcinoma
Renal Cell Carcinoma with Lung Metastases
Renal Pelvis Cancer
Retinoblastoma (Adult)
Retinoblastoma (Child)
Rhabdomyosarcoma
Richter's Syndrome
Rising PSA as an Indication of Recurrent Prostate Cancer

-S-

Salivary Gland Cancer including:
Adenoid cystic carcinoma
Mucoepidermoid carcinoma
Sarcoma of Bone
Sarcomatoid Carcinoma
Schwannoma, Malignant
Sebaceous Carcinoma
Sezary Syndrome
Sinus, Cavernous
Sinus Cancer
Sinus Cancer, Cavernous
Sinus Cancer, Endodermal
Sinus Cancer, Esthesioneuroblastoma
Sinus Cavity Cancer
Skin Cancer:
Basal Cell - nodular, nevoid, superficial, morpheaform (also termed aggressive-growth
BCC or infiltrative BCC), pigmented, cystic BCC, and fibroepithelioma of Pinkus
(FEP)
Merkel Cell
Squamous Cell - keratoacanthoma, periungual
Skin Metastases
Atypical fibroxanthoma
Actinic keratoses
Dermatofibroma
Desmoplastic trichoepithelioma
Microcystic adrexal carcinoma
Non-melanoma skin cancer
Verrucous carcinoma
Malignant Fibrous Histiocytoma
Dermatofibrosarcoma Protuberans
Bednar Tumor
Angiosarcoma
Kaposi's Sarcoma
Carcinoma Metastatic to Skin
Small Cell Bladder Cancer
Small Cell Lung Cancer
Small Cell Lung Cancer, Extensive Stage
Small Cell Lung Cancer, Limited Stage
Small Cell Lung Cancer with Brain Metastases
Small Cell Lung Cancer with Liver Metastases
Small Cleaved Cell Lymphoma
Small Intestine Cancer, including:
Adenocarcinoma
Carcinoid
Foregut
Hindgut
Midgut
Fibromatosis (Desmoid Tumor)
Gastrointestinal Stromal Tumor
Inflammatory Fibroid Polyps
Lymphoma
Burkitt's Lymphoma
Diffuse Large B-Cell Lymphoma
Mucosal-Associated Lymphoid Tissue Lymphoma
T-Cell Lymphomas of the Small Intestine
Myofibroblastic Tumor
Schwannoma
Simple Leiomyoma
Solitary Fibrous Tumor
True Leiomyoma
Soft Tissue Lymphoma
Soft Tissue Sarcoma:
Soft Tissue Sarcoma (Adult)
Soft Tissue Sarcoma (Child)
Soft Tissue Sarcoma, Angiosarcoma
Soft Tissue Sarcoma, Clear Cell
Soft Tissue Sarcoma, Dermatofibrosarcoma Protuberans
Soft Tissue Sarcoma, Hemangiopericytoma
Soft Tissue Sarcoma, Leiomyosarcoma
Soft Tissue Sarcoma, Liposarcoma
Soft Tissue Sarcoma, Malignant Fibrous Histiocytoma
Soft Tissue Sarcoma, Primitive Neuroectodermal (Adult)
Soft Tissue Sarcoma, Primitive Neuroectodermal (Child)
Soft Tissue Sarcoma, Rhabdomyosarcoma
Soft Tissue Sarcoma, Synovial
Synovial Sarcoma
Epithelioid Sarcoma:
Ewing's Sarcoma
Extraskeletal Cartilaginous Tumors
Extraskeletal Chondrosarcoma
Mesenchymal Chondrosarcoma
Myxoid Chondrosarcoma
Extraskeletal Osteosarcoma
Fibrohistiocytic Tumors
Atypical Fibroxanthoma
Angiomatoid Fibrous Histiocytoma
Dermatofibrosarcoma Protuberans
Giant Cell Fibroblastoma
Malignant Fibrous Histiocytoma
Giant Cell
Inflammatory
Myxoid
Storiform-Pleomorphic
Plexiform Fibrohistiocytic Tymor
Fibrous Tumors
Fibromatoses
Deep Fibromatoses
Abdominal Fibromatosis
Extraabdominal Fibromatosis
Intraabdominal Fibromatosis
Infantile Fibromatosis
Mesenteric Fibromatosis
Superficial Fibromatoses
Palmar
Penile (Peyronie's Fibromatosis)
Fibrosarcoma
Adult Fibrosarcoma
Inflammatory Fibrosarcoma
Infantile Fibrosarcoma
Inflammatory Myofibroblasic Tumor
Gastrointestinal Stromal Tumors
Kaposi's Sarcoma
Lipomatous Tumors
Atypical Lipoma
Liposarcoma
Dedifferentiated Liposarcoma
Myxoid (Round Cell) Liposarcoma
Pleomorphic Liposarcoma
Well-differentiated Liposarcoma
Inflammatory Liposarcoma
Lipoma-like Liposarcoma
Sclerosing Liposarcoma
Lymphangiosarcoma
Malignant Giant Cell Tumor of Tendon Sheath
Malignant Glomus Tumor (Glomangiosarcoma)
Malignant Extrarenal Rhabdoid Tumor
Malignant Hemangiopericytoma
Malignant Mesenchymoma
Neural Tumors
Malignant Peripheral Nerve Sheath Tumor (MPNST)
Malignant Triton Tumor
Epithelioid MPNST
Glandular MPNST
Reticulum Cell Sarcoma (Lymphoma of bone)
Skeletal Muscle Tumors
Ectomesenchymoma
Rhabdomyosarcoma
Alveolar Rhabdomyosarcoma
Botryoid Rhabdomyosarcoma
Embryonal Rhabdomyosarcoma
Pleomorphic Rhabdomyosarcoma
Spindle Cell Rhabdomyosarcoma
Small Round Cell Sarcoma
Histiocytosis
Metastatic Neuroblastoma
Metastatic Undifferentiated Carcinoma
Multiple Myeloma
Osteomyelitis
Small Cell Osteosarcoma
Smooth Muscle Tumors
Epithelioid Leiomyosarcoma
Leiomyosarcoma
Solitary Fibrous Tumor
Spindle Cell Hemangioendothelioma
Synovial Sarcoma
Biphasic Synovial Sarcoma
Monophasic Synovial Sarcoma
Spinal Cord Cancer
Spindle Cell Thyroid Cancer
Squamous Cell Cancer (Non-Skin)
Squamous Cell Lung Cancer
Squamous Cell Skin Cancer
Stem Cell Transplantation
Stomach Cancer, including:
Stomach Cancer, Adenocanthoma
Stomach Cancer, Adenocarcinoma
Stomach Cancer, Adenosquamous Carcinoma
Stomach Cancer, Gastric Carcinoid
Stomach Cancer, Gastric Leiomyosarcoma
Stomach Cancer, Gastric Lymphoma
Stomach Cancer, Gastric Plasmacytoma
Stomach Cancer, Linitis Plastica
Stomach Cancer, Scirrhous Carcinoma
Stomach Cancer, Signet Ring Cell
Stomach Cancer, Mucinous Adenocarcinoma
Stomach Cancer, Mixed adeno- and Choriocarcinoma
Stomach Cancer, Papillary Adenocarcinoma
Stomach Cancer, Squamous Cell Carcinoma
Stomach Cancer with Bone Metastases
Superficial Bladder Cancer
Sweat Glands Cancer

-T-

T-Cell Leukemia
T-Cell Lymphoma, Cutaneous
Testicular Cancer, Adenocarcinoma of the Rete Testis
Testicular Cancer, Carcinoma In Situ
Testicular Cancer, Choriocarcinoma
Testicular Cancer, Epidermoid Cyst
Testicular Cancer, Germ Cell
Testicular Cancer, Gonadoblastoma
Testicular Cancer, Granulosa Cell Tumors
Testicular Cancer, Leydig Cell Tumors
Testicular Cancer, Lymphoma
Testicular Cancer, Mesothelioma
Testicular Cancer, Metastatic Carcinoma
Testicular Cancer, Non-Seminoma:
Choriocarcinoma
Embryonal Carcinoma
Teratoma
Immature
Mature
With Malignant Differentiation
Yolk Sac
Testicular Cancer, Seminoma:
Anaplastic
Atypical
Typical
Testicular Cancer, Sarcomas
Testicular Cancer, Sertoli Cell Tumors:
Classic
Large Cell Calcifying
Sclerosing
Testicular Cancer, Stage I
Testicular Cancer, Stage II
Testicular Cancer, Stage III
Thalamus (Thalamic) Brain Tumor
Thymoma
Thymus Cancer, including:
Thymic Carcinoid
Thymic Carcinoma
Clear-Cell Carcinoma
Lymphoepithelioma-like Carcinoma
Mucoepidermoid Carcinoma
Sarcomatoid
Squamous Cell Carcinoma
Thymolipoma
Thymoma
Cortical
Medullary
Mixed
Thyroid Cancer:
Papillary thyroid carcinoma with papillary renal neoplasia (PTC-RCC)
Familial nonmedullary thyroid carcinoma (fNMTC)
Familial thyroid tumors with cell oxyphilia (TCO)
Papillary thyroid cancer without oxyphilia
Familial adenomatous polyposis (FAP)
Thyroid malignancy derived from follicular or parafollicular C cells
Medullary thyroid carcinoma (MTC)
Cowden disease
Thyroid Lymphoma
Tongue Cancer
Tongue Base Cancer
Transitional Cell Cencer of Bladder
Transitional Cell Cancer of Renal Pelvis
Transitional Cell Cancer of the Ureter
Transition Cell Cancer of the Urethra
Tuberous Sclerosis (Bourneville Disease)
Tylosis

-U-

Unknown Primary Cancer
Ureter Cancer
Urethra Cancer:
Adenocarcinoma
Clear Cell
Melanoma
Squamous Cell
Transitional Cell
Undifferentiated
Urethra Cancer, Carcinoma of Bulbomembranous Urethra
Urethra Cancer, Carcinoma of Distal Urethra
Urethra Cancer, Carcinoma of Prostatic Urethra
Urethra Cancer, Carcinoma of Proximal Urethra
Uterine Cancer
Uterine Cancer, Endometrial
Uterine Cancer, Carcinosarcoma
Uterine Cancer, Leiomyosarcoma
Uterine Cancer, Papillary Serous
Uterine Cancer, Sarcoma
Uterine Cancer, Stage I
Uterine Cancer, Stage II
Uterine Cancer, Stage III
Uterine Cancer, Stage IV
Uterine Sarcoma

-V-

Vagina Cancer:
Adenocarcinomas
Adenosquamous
Clear Cell
Mucinous
Papillaiy
Undifferentiated
Primary Vaginal Melanomas
Sarcoma
Embryonal Rhabdomyosarcoma (Sarcoma Botryoides)
Endometrial Stromal Sarcoma
Leiomyosarcoma
Malignant Mixed Mullerian Tumor
Small Cell Carcinoma
Squamous Cell Carcinoma
Keratinizing
Nonkeratinizing
Vaginal Intraepithelial Neoplasia
Verrucous Carcinoma
Vocal Cord Cancer
Vulva Cancer:
Adenocarcinoma
Anaplastic Carcinomas
Large Immature Cells
Spindle Sarcomatoid Cells
Small Cells
Bartholin's Gland Carcinoma
Adenocarcinoma
Adenoid Cystic Carcinoma
Squamous Cell Carcinoma
Transitional Cell Carcinoma
Basal Cell Carcinoma
Lichen Sclerosis
Malignant Melanoma
Paget's Lesions
Primary Mammary Adenocarcinoma
Sebaceous Carcinoma
Squamous Cell Carcinoma
Squamous Hyperplasia
Verrucous Carcinoma
Vulvar Intraepithelial Neoplasia
Vulvar Sarcoma
Angiosarcoma
Epithelioid Sarcoma
Leiomyosarcoma
Neurofibrosarcoma
Rhabdomyosarcoma

-W-

Waldenstrom's Macroglobulinemia
Warthin's Tumor
Wilms' Tumor (Child)

-Z-

Zollinger-Ellison Syndrome

### EXAMPLES

Patients with inoperable hepatocellular carcinoma (HCC), no prior systemic treatment, and Child-Pugh (CP) score of A or B, received oral sorafenib at 400 mg bid continuously in 4-week cycles. Tumor response was assessed every two cycles using modified WHO criteria and independent radiologic review. Plasma samples were collected to evaluate the pharmacokinetics (PK) of sorafenib. Biomarker assays were performed in selected patients. These included phospho-ERK levels via immunohistochemistry in pretreatment biopsies, plasma HER-2/neu analysis, plasma protein proteomics via mass spectrometry, and blood cell RNA expression patterns via Affymetrix GeneChip microarray analysis.

Results: Of 137 patients who were enrolled (M:F=97:40; median age 69 years [range 28-86]), 98 (72%) had CP A and 38 (28%) CP B. Based on independent radiologic review, three (2.2%) patients achieved a partial response, eight (5.8%) achieved a minor response, and 46 (33.6%) had stable disease for at least 16 weeks as best response. Investigator-assessed median TTP was 4.2 months, and median overall survival was 9.2 months. The most common grade ¾ drug-related toxicities which led to dose delay and/or modification were diarrhea (4.4%), hand-foot skin reaction (5.1%) and fatigue (5.8%). There were no pharmacokinetic differences between CP A and B patients. A correlation was found between pretreatment tumor phospho-ERK levels and patient response.

1. A method of treating a cancer listed in Table 1 below in a subject in need thereof, comprising, administering an effective amount of sorafenib to said subject having said cancer
TABLE 1
Adenocarcinoma
Adenocarcinoma Lung Cancer
Adenocystic Carcinoma
Adenoid Cystic Breast Cancer
Adenoid Cystic Carcinoma
Adrenal Gland Cancer:
Adrenal Gland Cancer - Cortical Adenoma
Adrenal Gland Cancer - Cortical Carcinoma
AIDS related disorders:
AIDS-related disorders - Kaposi's sarcoma
AIDS-related disorders, - lymphoma
Advanced Breast Cancer
Aggressive Breast Cancer
Ameloblastoma
Anal (Anus) Cancer, including:
Anal Cancer - Early-Stage (Superficial)
Anal Cancer - Intermediate-Stage I
Anal Cancer - Intermediate-Stage II
Anal Cancer - Intermediate-Stage III
Anal Cancer - Metastatic, Advanced-Stage IV
Anal Cancer - Adenocarcinomas
Anal Cancer - Intraepithelial Neoplasia
Anal Cancer - Bowen's Disease
Anal Cancer - Paget's Disease
Anal Cancer - Small Cell Cancers
Anal Cancer - Squamous Cell Carcinoma
Angiosarcoma
Appendix Cancer (Appendiceal Cancer)
Ascites, Malignant
Astrocytoma
Barrett's Esophagus
Bartholin Gland Cancer
Basal Cell Carcinoma
Basal Cell Skin Cancer
B-Cell Lymphoma
Bilateral Breast Cancer
Bile Duct Cancer
Bladder Cancer:
Bladder Cancer - Superficial
Bladder Cancer - Muscle-Invasive
Bladder Cancer - Small Cell
Bladder Cancer - Metastases
Bone Cancer:
Bone Cancer - Chordoma
Bone Cancer - Chondrosarcoma
Bone Cancer - Ewing's Sarcoma (Child)
Bone Cancer - Giant Cell
Bone Cancer - Mesenchymal Chondrosarcoma
Bone Cancer - Osteosarcoma
Bone Cancer - Bone Marrow Transplantation
Bone Cancer - Bone Metastases
Bowen's Disease
Borderline Ovarian Cancer
Brain Tumors:
Brain Tumors - Adult
Brain Tumors - Child
Brain Tumors - Astrocytoma
Brain Tumors - Brainstem
Brain Tumors - Cavernous Sinus
Brain Tumors - Chondroma
Brain Tumors - Ependymoma
Brain Tumors - Glioblastoma
Brain Tumors - Glioma
Brain Tumors - Gliomatosis
Brain Tumors - Hemangiopericytoma
Brain Tumors - Medulloblastoma
Brain Tumors - Oligodendroglioma
Brain Tumors - Pineoblastoma/Pinealoma
Brain Tumors - Primary Central Nervous System Lymphoma
Brain Tumors - Thalamic
Brain Tumors - Brainstem Glioma
Breast Cancers Including:
Breast Cancer - Adenoid Cystic
Breast Cancer - Advanced
Breast Cancer - Aggressive
Breast Cancer - Apocrine Carcinoma
Breast Cancer - Bilateral (in both breasts)
Breast Cancer - Comedo Type
Breast Cancer - Ductal Carcinoma In Situ
Breast Cancer - Ductal Carcinoma Invasive
Breast Cancer - Early Stage
Breast Cancer - Estrogen-Negative
Breast Cancer - High-Risk
Breast Cancer - Inflammatory
Breast Cancer - in Pregnancy
Breast Cancer - Lobular, Invasive
Breast Cancer - Lobular Carcinoma In situ (LCIS)
Breast Cancer - Lymphoma
Breast Cancer - Medullary
Breast Cancer - Microinvasive
Breast Cancer - Mucinous
Breast Cancer - Node-Negative
Breast Cancer - Node-Positive
Breast Cancer, Nonepithelial Neoplasms:
Breast Cancer - Nonepithelial Neoplasms - Phyllodes Sarcomas
Breast Cancer - Nonepithelial Neoplasms - Phyllodes Tumors
Breast Cancer - Occult with Lymphatic Metastasis
Breast Cancer - Papillary
Breast Cancer - Postmenopausal
Breast Cancer - Premenopausal
Breast Cancer - Recurrent
Breast Cancer - Sarcoma
Breast Cancer - Secretory (juvenile) Carcinoma
Breast Cancer - Squamous
Breast Cancer - Stage I
Breast Cancer - Stage II
Breast Cancer - Stage III
Breast Cancer - Stage IV
Breast Cancer - Tubular
Breast Cancer - Ulcerating
Breast Cancer with Bladder Metastases
Breast Cancer with Bone Metastases
Breast Cancer with Bone and Lung Metastases
Breast Cancer with Brain Metastases
Breast Cancer with Brain and Liver Metastases
Breast Cancer with Central Nervous System Metastases
Breast Cancer with Liver Metastases
Breast Cancer with Lung Metastases
Breast Cancer with Lymphangitic Metastasis
Breast Cancer with Lymphatic Metastases
Breast Cancer with Lymph Node Metastases
Breast Cancer with Meningeal Metastases
Breast Cancer with Pericardial Effusion
Breast Cancer with Pleura Metastases
Breast Cancer with Pleural Effusion
Breast Cancer with Skin Metastases
Breast Cancer with Spine Metastases
Breast Cancer with Stomach Metastases
Breast Cancer - Breast Reconstruction
Breast Cancer - Comedo Type Breast Cancer
Breast Cancer - Early Stage Breast Cancer
Breast Cancer - Estrogen-Negative Breast Cancer
Breast Cancer - Inflammatory Breast Cancer
Breast Cancer - Invasive Breast Cancer
Breast Cancer - Lobular Breast Cancer
Breast Cancer - Medullary Breast Cancer
Breast Cancer - Metachronous Bilateral
Breast Cancer - Microinvasive Breast Cancer
Breast Cancer - Mucinous Breast Cancer
Breast Cancer - Node-Negative Breast Cancer
Breast Cancer - Node-Positive Breast Cancer
Breast Cancer - Occult Breast Cancer with Lymphatic Metastasis
Breast Cancer - Paget's Disease of the Nipple
Breast Cancer - Papillary Breast Cancer
Breast Cancer - Recurrent Breast Cancer
Breast Cancer - Sarcoma of the Breast
Breast Cancer - Squamous Cell Breast Cancer
Breast Cancer - Synchronous Bilateral Breast Cancer
Breast Cancer - Tubular Breast Cancer
Breast Cancer - Ulcerating Breast Cancer
Bronchial Cancer
Bronchioalveolar Lung Cancer
Buttock Cancer
Cancer of Unknown Primary Site (CUPS)
Carcinoma of Unknown Primary
Carcinoid of the Appendix
Carcinoid of the Colon
Carcinoid of the Pancreas
Carcinoid of the Rectum
Carcinoid of the Stomach
Carcinoid Syndrome
Carcinoid Tumor
Carcinosarcoma of the Ovary
Carcinosarcoma of the Uterus
Carney's Triad
Cavernous Sinus Cancer
Cecum Cancer
Central Nervous System Lymphoma
Central Nervous System Cancers:
Central Nervous System Cancers - Medulloblastoma
Central Nervous System Cancers - Astrocytomas
Central Nervous System Cancers - Gliomas - Optic, Chiasmal, and
Hypothalamic
Central Nervous System Cancers - Cystic Cerebellar Astrocytomas
Central Nervous System Cancers - Diffuse, Fibrillary Astrocytomas - e.g.,
World Health Organization (WHO) Grade II
Central Nervous System Cancers - Astrocytoma, e.g., WHO grade III
Anaplastic Astrocytoma, and WHO grade IV
Central Nervous System Cancers - Glioblastoma (GBM)
Central Nervous System Cancers - Pilocytic Astrocytoma
Central Nervous System Cancers - Pleomorphic Xanthoastrocytoma
Central Nervous System Cancers - Subependymal Giant Cell
Astrocytomas
Central Nervous System Cancers - Oligodendroglioma- Anaplastic,
differentiated
Central Nervous System Cancers - Oligoastrocytoma- Anaplastic,
differentiated
Central Nervous System Cancers - Ependymoma- Ellular, Papillary, Clear
Cell, Tanycytic, Myxopapillary
Central Nervous System Cancers - Choroid Plexus Tumor
Central Nervous System Cancers - Meningioma- Atypical and Malignant,
Parasagittal, Falx, Olfactory Groove, Tuberculum Sellae,
Sphenoid Ridge, Cerebellopontine Angle
Central Nervous System Cancers - Schwannoma
Central Nervous System Cancers - Neurofibromas
Central Nervous System Cancers - Hemangioblastomas
Central Nervous System Cancers - Atypical Teratoid/Rhabdoid Tumors
Central Nervous System Cancers - Gorlin's, Turcot, and Li-Fraumeni
syndromes
Central Nervous System Cancers - Supratentorial Parenchymal tumors
Central Nervous System Cancers - Medulloepithelioma
Central Nervous System Cancers - Neuroblastoma
Central Nervous System Cancers - Ependymoblastoma
Central Nervous System Cancers - Pineoblastoma
Central Nervous System Cancers - Pineal Parenchymal Tumors
Central Nervous System Cancers - Germinomas
Central Nervous System Cancers - Pituitary Adenoma
Central Nervous System Cancers - Craniopharyngioma
Central Nervous System Cancers - Acoustic Neuromas (Vestibular
Schwannomas)
Central Nervous System Cancers - Glomus Jugulare tumors
(Paragangliomas)
Central Nervous System Cancers - Chordoma and Chondrosarcoma
Central Nervous System Cancers - Hemangioblastomas
Central Nervous System Cancers - Choroid Plexus Papilloma and
Carcinoma
Central Nervous System Cancers - Spinal Axis Tumors
Centrocytic Lymphoma
Cerebral Astrocytoma
Cervix Cancer (Cervical Cancer):
Cervix Cancer (Cervical Cancer), Adenocarcinoma In Situ:
Cervical Cancer, Adenocarcinoma In Situ - Adenoid Basal Carcinoma
Cervical Cancer, Adenocarcinoma In Situ - Adenoma Malignum
Cervical Cancer, Adenocarcinoma In Situ - Adenosquamous
Carcinoma
Cervical Cancer, Adenocarcinoma In Situ - Glassy Cell Carcinoma
Cervical Cancer, Adenocarcinoma In Situ - Papillary Serous
Carcinoma
Cervix Cancer (Cervical Cancer), Anaplastic Small Cell Carcinoma
Cervix Cancer (Cervical Cancer), Cervical Intraepithelial Neoplasia
Cervix Cancer (Cervical Cancer), Malignant Mixed M?u♭llerian Tumors
Squamous Cell Carcinoma:
Squamous Cell Carcinoma - Large Cell Keratinizing
Squamous Cell Carcinoma - Large Call Nonkeratinizing
Squamous Cell Carcinoma - Papillary
Squamous Cell Carcinoma - Sarcomatoid Squamous Carcinoma
Squamous Cell Carcinoma - Small Cell Carcinoma
Squamous Cell Carcinoma - Verrucous Carcinoma
Chest Tumors (Mediastinum Tumors)
Chiasmal Glioma
Childhood Cancers
Chondroma, Intracranial
Chordoma
Chondrosarcoma:
Chondrosarcoma - Central
Chondrosarcoma - Clear Cell
Chondrosarcoma - Differentiated
Chondrosarcoma - Mesenchymal
Chondrosarcoma - Peripheral
Chorioadenoma Destruens
Choriocarcinoma:
Choriocarcinoma, Syncytiotrophoblast
Choriocarcinoma, Cytotrophoblast
Choroid Plexus Carcinoma
Choroidal Melanoma
Chronic Leukemia
Chronic Lymphocytic Leukemia (CLL)
Chronic Myelogenous Leukemia (CML)
Clear Cell Ovarian Cancer
Clear Cell Sarcoma
CNS Lymphoma
Colon Cancer Including:
Colon Cancer - Amine Precursor Uptake and Decarboxylation Tumor
Colon Cancer - Cecum
Colon Cancer - Lymphoma
Colon Cancer - Lymphoma - B-Cell
Colon Cancer - Lymphoma - T-Cell
Colon Cancer - Hamartomatous Polyposis Syndrome
Colon Cancer - Hereditary Nonpolyposis Colorectal Cancer
Colon Cancer - Mucinous
Colon Cancer, Neuroendocrine Carcinoma:
Colon Cancer - Neuroendocrine Carcinoma -
Extrapulmonary Small Cell
Colon Cancer - Neuroendocrine Carcinoma - High-grade
Colon Cancer - Neuroendocrine Carcinoma - Oat Cell
Colon Cancer - Neuroendocrine Carcinoma - Well-differentiated Carcinoid
Colon Cancer - Recurrent
Colon Cancer - Stage A
Colon Cancer - Stage B
Colon Cancer - Stage C
Colon Cancer - Stage D
Colon Cancer - Stage I (Dukes' A)
Colon Cancer - Stage II (Dukes' B)
Colon Cancer - Stage III (Dukes' C)
Colon Cancer - Stage IV
Colon Cancer with Bone Metastases
Colon Cancer with Brain Metastases
Colon Cancer with Buttocks Metastases
Colon Cancer with Liver Metastases
Colon Cancer with Liver and Lung Metastases
Colon Cancer with Lung Metastases
Colon Cancer with Ovarian Metastases
Colon Cancer with Skin Metastases
Colon Cancer - Adenocarcinoma of the Colon - Mucinous (Colloid)
Colon Cancer - Adenocarcinoma of the Colon - Signet Ring
Colon Cancer - Adenomatous Polyps
Colon Cancer - Polyps
Colorectal Cancer, Colon Cancer
Craniopharyngioma
Cutaneous T-Cell Lymphoma
DCIS - Ductal Carcinoma In Situ
Dermatofibrosarcoma Protuberans
Desmoid Tumor
Desmoplastic Tumor
Diffuse Lymphoma
Diffuse, Large-Cell Lymphoma
Ductal Carcinoma in Situ (DCIS)
Duodenal Cancer
Dysgerminoma
-E-
Early Stage Prostate Cancer
Endochodroma
Endocrine Cancer:
Endocrine Cancer - Multiple Endocrine Neoplasia Type 1 (MEN 1)
Endocrine Cancer - Multiple Endocrine Neoplasia Type 2 (MEN 2)
Endocrine Cancer - MEN 2A
Endocrine Cancer - MEN 2B
Endocrine Cancer - Familial Non-MEN MTC (FMTC)
Endocrine Cancer - Sporadic MTC
Endocrine Cancer - Sporadic Thyroid Neoplasms
Endocrine Cancer - Papillary Thyroid Carcinoma
Endocrine Cancer - Follicular Adenomas
Endometrial Cancer:
Endometrial Cancer - Adenosarcoma
Endometrial Cancer - Epithelial Tumors
Endometrial Cancer - Epithelial Tumors - Clear Cell
Endometrial Cancer - Epithelial Tumors - Endometrioid
Endometrial Cancer - Epithelial Tumors - Mucinous
Endometrial Cancer - Epithelial Tumors - Papillary Endometrioid
Endometrial Cancer - Epithelial Tumors - Papillary Serous
Endometrial Cancer - Epithelial Tumors - Malignant Mixed Mullerian
Endometrial Cancer - Mesenchymal Tumors:
Endometrial Cancer - Mesenchymal Tumors - Endometrial Stromal
Sarcoma
Endometrial Cancer - Mesenchymal Tumors - Leiomyosarcoma
Ependymoma
Esophagus Cancer (Esophageal Cancer), Including:
Esophagus Cancer (Esophageal Cancer) - Adenoid Cystic Carcinoma
Esophagus Cancer (Esophageal Cancer) - Mucoepidermoid Carcinoma
Esophagus Cancer of the Gastroesophageal Junction
Esthesioneuroblastoma
Ewing's Sarcoma
Extensive Stage Small Cell Lung Cancer
Extrahepatic Bile Duct Cancer
Extraskeletal Chondrosarcoma
Extraskeletal Ewing's Sarcoma
Extraskeletal Osteosarcoma
Eye Metastases
Eye Cancer
Eye Cancer, Lymphoma
-F-
Fallopian Tube Cancer Including:
Fallopian Tube Cancer - Adenocarcinomas
Fallopian Tube Cancer - Eiomyosarcomas
Fallopian Tube Cancer - Transitional cell carcinomas
Fallopian Tube Cancer - Stage I
Fallopian Tube Cancer - Stage II
Fallopian Tube Cancer - Stage III
Fallopian Tube Cancer - Stage IV
Fallopian Tube Cancer - Carcinoma
Fallopian Tube Cancer - Ovarian Cancer
Familial Adenomatous Polyposis
Fibrosarcoma - See Soft Tissue Sarcomas
Fibrous Histiocytoma
Follicular Lymphoma
Follicular, Small-Cleaved Cell Lymphoma
Gardner's Syndrome
Gallbladder Cancer, Including:
Gallbladder Cancer - Adenocarcinoma
Gallbladder Cancer - Mucinous Adenocarcinoma
Gallbladder Cancer - Papillary Adenocarcinoma
Gallbladder Cancer - Sarcoma
Gallbladder Cancer - Squamous Cell Carcinoma
Gastric Cancer
Gastrinoma
Gastro-Esophageal (GE Junction) Cancer
Gastrointestinal Lymphoma
Gastrointestinal Stromal Tumors (GST):
Gastrointestinal Stromal Tumors - Autonomic Nerve Tumor
Gastrointestinal Stromal Tumors - Leiomyoblastoma
Gastrointestinal Stromal Tumors - Leiomyosarcoma
Gastrointestinal Stromal Tumors - Pacemaker Call Tumor
Gastrointestinal Stromal Tumors - Plexosarcoma
Neurofibrosarcoma
GE Junction Esophageal Cancer
Germ Cell Tumor:
Germ Cell Tumor - Retroperitoneal Tumor
Germ Cell Tumor - Mediastinum Tumor
Germ Cell Tumor - Undescended Testicular Cancer
Gestational Trophoblastic Disease
Giant Cell Bone Tumor
Glioblastoma
Glioma
Gliomatosis
Gonadal Mesothelioma
Gorlin's Syndrome (Nevoid Basal Cell Carcinoma Syndrome)
Granular Lymphocytic Lymphoma
Granulocytic Leukemia
Granulosa Cell Ovarian Cancer
Hairy Cell Leukemia
Head and Neck Cancer
Head and Neck Cancer, Adenoid Cystic
Head and Neck Cancer, Squamous Cell
Hemangioendothelioma
Hemangiopericytoma
Hepatocelluar Carcinoma
High-Grade Lymphoma
High-Risk Breast Cancer
Histiocytoma, Malignant
Hodgkin's Disease
Hodgkin's Disease in Pregnancy
Hormone-Refractory Prostate Cancer
In Situ Breast Cancer
Insulinoma
Intermediate-Grade Lymphoma
Intraocular Melanoma
Invasive Lobular Breast Cancer
Islet Cell Pancreatic Cancer
Kaposi's Sarcoma
Kidney Cancer (Renal Cell Carcinoma), Including:
Kidney Cancer (Renal Cell Carcinoma) - Birt-Hogg-Dubé (BHD)
Kidney Cancer (Renal Cell Carcinoma) - Chromophobe Renal Carcinoma
Kidney Cancer (Renal Cell Carcinoma) - Clear Cell Renal Carcinoma
Kidney Cancer (Renal Cell Carcinoma) - Hereditary Papillary Renal Cell
Carcinoma (HPRC)
Kidney Cancer (Renal Cell Carcinoma) - Hereditary Leiomyomatosis
Renal Cell Carcinoma (HLRCC)
Kidney Cancer (Renal Cell Carcinoma) - Onocytoma Renal Carcinoma
Kidney Cancer (Renal Cell Carcinoma) - Papillary Renal Carcinoma
Kidney Cancer (Renal Cell Carcinoma) - Papillary Renal Carcinoma,
Type 1
Kidney Cancer (Renal Cell Carcinoma) - Papillary Renal Carcinoma,
Type 2
Kidney Cancer (Renal Cell Carcinoma) - Von Hippel-Lindau (VHL) Clear
Cell Renal Cell Carcinoma
Kidney Cancer with Lung Metastases
Krukenberg Tumor
Large-Cell Lung Cancer
Large-Cell Lymphoma
Large-Cell, Diffuse Lymphoma
Larynx Cancer (Laryngeal Cancer)
Leiomyosarcoma:
Leiomyosarcoma - Foot
Leiomyosarcoma - Pulmonary Artery
Leiomyosarcoma - Vena Cava
Leiomyosarcoma - Uterus
Lentigo Maligna
Leukemia:
Leukemia - Acute
Leukemia - Acute Lymphocytic (ALL)
Leukemia - Acute Megakaryoblastic leukemia
Leukemia - Acute Monocytic (AML)
Leukemia - Acute Myelogenous (AML)
Leukemia - Acute Myeloid (AML)
Leukemia - Acute Promyelocytic (APL)
Leukemia - Chronic
Leukemia - B-Cell Chronic Lymphocytic Leukemia
Leukemia - Chronic granulocytic leukemia
Leukemia - Chronic Lymphocytic (CLL)
Leukemia - Chronic Myelogenous
Leukemia - Chronic Myeloid (CML)
Leukemia - Chronic Myelomonocytic (CMML)
Leukemia - CML, Philadelphia-Negative
Leukemia - CMML
Leukemia - Cutis
Leukemia - Granulocytic
Leukemia - Granulocytic Sarcomas and Chloromas (Leukemic tumors)
Leukemia - Hairy Cell
Leukemia - Infant Acute Megakaryoblastic Leukemia
Leukemia - Large Granular Lymphocyte Leukemia
Leukemia - Lymphoblastic
Leukemia - Mature B-Cell Acute Lymphocytic Leukemia
Leukemia - Precursor B-Lymphoblastic Leukemia
Leukemia - Philadelphia Chromosome-Positive Acute Lymphocytic
Leukemia
Leukemia - Prolymphocytic
Leukemia - Promyelocytic
Leukemia - Secondary Myeloid Leukemia
Leukemia - T-Cel
Li-Fraumeni syndrome
Linitis Plastica
Limited Stage Small Cell Lung Cancer
Lip Cancer
Liposarcoma
Liver Cancer Including:
Liver Cancer - Angiosarcoma
Liver Cancer - Cholangiocarcinoma (Bile Duct Cancer):
Liver Cancer - Cholangiocarcinoma (Bile Duct Cancer) - Distal
Liver Cancer - Cholangiocarcinoma (Bile Duct Cancer) - Hilar
(Klatskin's Tumors)
Liver Cancer - Cholangiocarcinoma (Bile Duct Cancer) - Intrahepatic
Liver Cancer - Cholangiocellular Carcinoma
Liver Cancer - Emangioendotheliomas
Liver Cancer - Epithelioid Hemangioendothelioma
Liver Cancer - Fibrosarcoma
Liver Cancer - Leiomyosarcoma
Liver Cancer - Hemangioendothelioma
Liver Cancer - Hemangiosarcoma
Liver Cancer - Hepatic Cystadenocarcinoma
Liver Cancer - Hepatoblastoma
Liver Cancer - Hepatocellular Carcinoma (Liver Cell Carcinoma):
Liver Cancer - Hepatocellular Carcinoma (Liver Cell Carcinoma) -
Carcinosarcoma
Liver Cancer - Hepatocellular Carcinoma (Liver Cell Carcinoma) -
Childhood
Liver Cancer - Hepatocellular Carcinoma (Liver Cell Carcinoma) - Clear
Cell
Liver Cancer - Hepatocellular Carcinoma (Liver Cell Carcinoma) -
Combined
Liver Cancer - Hepatocellular Carcinoma (Liver Cell Carcinoma) -
Fibrolamellar
Liver Cancer - Hepatocellular Carcinoma (Liver Cell Carcinoma) - Giant
Cell
Liver Cancer - Hepatocellular Carcinoma (Liver Cell Carcinoma) -
Sclerosing, Hepatoblastoma
Liver Cancer - Hepatocellular Carcinoma (Liver Cell Carcinoma) - Spindle
Cell
Liver Cancer - Histiocytoma
Liver Cancer - Lymphoma
Liver Cancer - Malignant Fibrous
Liver Cancer - Malignant Schwannoma
Liver Cancer - Mesenchymal sarcoma
Liver Cancer - Metastases
Liver Cancer - Mixed Hepatocellular Cholangiocarcinoma
Liver Cancer - Osteosacoma
Liver Cancer - Rhabdomyosarcoma
Liver Cancer - Stage I
Liver Cancer - Stage II
Liver Cancer - Stage III
Liver Cancer - Stage IV
Liver Cancer - Recurrent Liver Cancer
Liver Cancer - Undifferentiated Liver Cancer
Lobular Carcinoma in Situ (LCIS)
Lobular Hyperplasia
Low-Grade Lymphoma
Lung Cancer:
Lung Cancer - Adenocarcinoma
Lung Cancer - Bronchioalveolar
Lung Cancer - Cystic Mucinous
Lung Cancer - Extrapulmonary Small Cell Anaplastic Carcinoma -
Limited
Lung Cancer - Extrapulmonary Small Cell Anaplastic Carcinoma -
Extensive
Lung Cancer - Germ Cell
Lung Cancer - Giant Cell
Lung Cancer - Large-Cell
Lung Cancer - Mucinous
Lung Cancer - Non-Small Cell Lung Cancer - Including:
Lung Cancer - Non-Small Cell Lung Cancer - (NSCLC)
Lung Cancer - Non-Small Cell Lung Cancer with Bone Metastases
Lung Cancer - Non-Small Cell Lung Cancer with Brain Metastases
Lung Cancer - Non-Small Cell Lung Cancer with Liver Metastases
Lung Cancer - Non-Small Cell Lung Cancer with Lymph Node Metastases
Lung Cancer - Non-Small Cell Lung Cancer with Skin Metastases
Lung Cancer - Non-Small Cell Lung Cancer - Stage I
Lung Cancer - Non-Small Cell Lung Cancer - Stage IIA
Lung Cancer - Non-Small Cell Lung Cancer - Stage IIB
Lung Cancer - Non-Small Cell Lung Cancer - Stage IIIA
Lung Cancer - Non-Small Cell Lung Cancer - Stage IIIB
Lung Cancer - Non-Small Cell Lung Cancer - Stage IV
Lung Cancer - Non-Small Cell Lung Cancer - Recurrent
Lung Cancer - Adenocarcinomas:
Lung Cancer - Adenocarcinomas - Acinar
Lung Cancer - Adenocarcinomas, Bronchioloalveolar Carcinoma
Lung Cancer - Adenocarcinomas, Bronchioloalveolar Carcinoma,
Nonmucinous (Clara Cell/type II Pneumocyte Type)
Lung Cancer - Adenocarcinomas, Bronchioloalveolar Carcinoma,
Mixed Mucinous and Nonmucinous/Indeterminate cell
type
Lung Cancer - Adenocarcinomas, Bronchioloalveolar Carcinoma,
Mucinous (Goblet Cell) type
Lung Cancer - Adenocarcinomas - Clear Cell Adenocarcinoma
Lung Cancer - Adenocarcinomas - Mucinous (Colloid)
Adenocarcinoma
Lung Cancer - Adenocarcinomas - Mucinous Cystadenocarcinoma
Lung Cancer - Adenocarcinomas - Papillary
Lung Cancer - Adenocarcinomas - Signet-Ring Adenocarcinoma
Adenocarcinomas - Solid Adenocarcinoma with Mucin
Formation
Lung Cancer - Adenocarcinomas - Well-differentiated Fetal
Adenocarcinoma
Adenosquamous Carcinoma
Atypical Adenomatous Hyperplasia
Carcinoid Tumors, Atypical
Carcinoid Tumors, Typical
Carcinosarcoma
Diffuse Idiopathic Pulmonary Neuroendocrine Cell Hyperplasia
Epidermoid Carcinoma
Giant Cell Carcinoma
Large Cell Carcinoma:
Large Cell Carcinoma - Basaloid Carcinoma
Large Cell Carcinoma - Clear Cell Carcinoma
Large Cell Carcinoma - Combined Large Cell Neuroendocrine
Carcinoma
Large Cell Carcinoma - Large Cell Carcinoma with Rhabdoid
Phenotype
Large Cell Carcinoma - Large Cell Neuroendocrine Carcinoma
Large Cell Carcinoma - Lymphoepithelioma-like Carcinoma
Pleomorphic Carcinoma
Pulmonary Blastoma
Spindle Cell Carcinoma
Squamous Cell Carcinomas:
Squamous Cell Carcinomas - Basaloid
Squamous Cell Carcinomas - Clear Cell
Squamous Cell Carcinomas - Papillary
Squamous Cell Carcinomas - Small Cell
Lung Cancer, Squamous Dysplasia/Carcinoma in situ
Lung Cancer, Superior Sulcus Tumors (Pancoast tumors)
Lung Cancer, Unclassified
Lung Cancer, Recurrent
Lung Cancer, Small Cell:
Lung Cancer - Small Cell - Combined small cell carcinoma
Lung Cancer - Small Cell - Mixed small cell/large cell carcinoma
Lung Cancer - Small Cell - Pure small cell
Lung Cancer - Small Cell - Extensive Stage
Lung Cancer - Small Cell - Limited Stage
Lung Cancer - Small Cell with Brain Metastases
Lung Cancer, Squamous
Lung Cancer, Stage I
Lung Cancer, Stage II
Lung Cancer, Stage III
Lung Cancer, Stage IIIA
Lung Cancer, Stage IIIB
Lung Cancer, Stage IV
Lung Cancer with Adrenal Gland Metastases
Lung Cancer with Bone Metastases
Lung Cancer with Brain Metastases
Lung Cancer with Liver Metastases
Lung Cancer with Lymph Node Metastases
Lung Cancer with Ovarian Metastases
Lung Cancer with Skin Metastases
Lung Metastases
Lymph Node Excision
Lymphangitic Metastasis
Lymph Node Metastases
Lymphoblastic Leukemia
Lymphoblastic Lymphoma
Lymphocytic Lymphoma
Lymphoma:
Lymphoma - Lymphoblastic Lymphoma
Lymphoma - Lymphocytic Lymphoma
Lymphoma - Lymphocytic, Large Granular
Lymphoma - Lymphocytic, Small
Lymphoma - AIDS Related
Lymphoma - Anaplastic
Lymphoma - Anaplastic Large Cell (ALCL)
Lymphoma - Breast
Lymphoma - B-Cell
Lymphoma - B-Cell Large- Lymphomatoid Granulomatosis Type
Lymphoma - B-Cell Large Diffuse
Lymphoma - B-Cell Monocytoid
Lymphoma - Central Nervous System
Lymphoma - Cutaneous T-Cell:
Lymphoma - Cutaneous T-Cell - Mycosis Fungoides, Agetoid Reticulosis
or Woringer-Kolopp disease
Lymphoma, Granulomatous slack skin:
Lymphoma - Granulomatous slack skin - Lymphomatoid
Lymphoma - Granulomatous slack skin - Papulosis
Lymphoma - Granulomatous slack skin - Primary
Lymphoma - Granulomatous slack skin - Cutaneous
Lymphoma - CD30+ Large T-cell Lymphoma
Lymphoma - CD56+ “Nasal-Type” NK/T-Cell Lymphoma
Lymphoma - Diffuse
Lymphoma - Diffuse, Large-Cell
Lymphoma - Enteropathy-Type T-Cell Lymphoma
Lymphoma - Eye
Lymphoma - Follicular
Lymphoma - Follicular, Small-Cleaved Cell
Lymphoma - Gastrointestinal
Lymphoma - Granular, Lymphocytic
Lymphoma - Hepatosplenic T-Cell Lymphoma
Lymphoma - High-Grade
Lymphoma - Hodgkin's Disease:
Lymphoma - Hodgkin's Disease - Paragranuloma
Lymphoma - Hodgkin's Disease - Granuloma
Lymphoma - Hodgkin's Disease - Sarcoma
Lymphoma - Hodgkin's Disease - Nodular Lymphocyte-Predominant
Hodgkin's Lymphoma
Lymphoma - Hodgkin's Disease - Nodular Lymphocyte-Predominant
Hodgkin's Lymphoma
Lymphoma - Hodgkin's disease - Mixed-Cellularity Hodgkin's
Lymphoma
Lymphoma - Hodgkin's disease - Nodular Sclerosis Hodgkin's
Lymphoma
Lymphoma - Hodgkin's disease - Lymphocyte-Depleted Hodgkin's
Lymphoma
Lymphoma - Hodgkin's disease - Lymphocyte-Predominant Hodgkin's
Lymphoma
Lymphoma - Hodgkin's disease - Lymphocyte-Rich Classic Hodgkin's
Lymphoma
Lymphoma - Intermediate Grade
Lymphoma - Hodgkin's disease, Intravascular Large B-Cell Lymphoma
Lymphoma - Large-Cell
Lymphoma - Large-Cell, Diffuse
Lymphoma - Low-Grade
Lymphoma - Hodgkin's disease, Low-Grade B-Cell Lymphoma of
Mucosa-Associated Lymphoid Tissue
Lymphoma - Hodgkin's disease, Lymphoplasmacytic lymphoma
Lymphoma - MALT
Lymphoma - Mantle Cell
Lymphoma - Non-Hodgkin's, Including:
Lymphoma - Non-Hodgkin's - High Grade
Lymphoma - Non-Hodgkin's - Intermediate Grade
Lymphoma - Non-Hodgkin's - Low Grade
Lymphoma - Non-Hodgkin's - Burkitt's & Atypical Burkitt's
Lymphoma - Non-Hodgkin's - Diffuse
Lymphoma - Non-Hodgkin's - Non-Burkitt's
Lymphoma - Non-Hodgkin's - T-Cell, Cutaneous
Lymphoma - Non-Hodgkin's - Immunoblastic Diffuse
Lymphoma - Non-Hodgkin's - Large Cell Follicular
Lymphoma - Non-Hodgkin's - Mixed Cell Diffuse
Lymphoma - Non-Hodgkin's - Small Cleaved Cell Follicular
Lymphoma - Non-Hodgkin's - Small Lymphocytic
Lymphoma - Non-Hodgkin's - Mixed Follicular
Lymphoma - Non-Hodgkin's - Small Cleaved Cell Diffuse,
Lymphoma - Non-Hodgkin's - Intermediately Differentiated Diffuse
Lymphoma - Non-Hodgkin's - Cutaneous T-cell
Lymphoma, Orbital
Lymphoma, Peripheral T-Cell
Precursor B-Lymphoblastic Lymphoma
Lymphoma, Primary Central Nervous System
Primary Effusion Lymphoma
Primary leptomeningeal lymphoma
Primary Mediastinal (Thymic) Large B-Cell LymphomaPrimary spinal
cord lymphoma
Richter's lymphoma
Lymphoma, Small Cleaved Cell
Lymphoma, Soft tissue
Splenic Marginal Zone Lymphoma with or without Villous Lymphocytes
Subcutaneous Panniculitis-Like T-Cell Lymphoma
Lymphoma, T-Cell
Lymphoma, T-Cell, Cutaneous
T-Cell- Rich/Histiocyte-Rich Large B-Cell Lymphoma
Lymphoma, Thyroid
Malignant Ascites
Malignant Carcinoid Syndrome:
Malignant Carcinoid Syndrome - Atypical
Malignant Carcinoid Syndrome - Typical
Malignant Hemangiopericytoma
Malignant Fibrous Histiocytoma
Malignant Peripheral Nerve Sheath Tumor
Malignant Pleural Effusion
Malignant Pleural Mesothelioma
Malignant Schwannoma
MALT Lymphoma
Mantle Cell Lymphoma
Mediastinal Neoplasms, including:
Mediastinal Neoplasms - Chemodectoma (Paraganglioma)
Mediastinal Neoplasms - Choriocarcinoma
Mediastinal Neoplasms - Endodermal Sinus Tumors (Yolk Sac Tumors)
Mediastinal Neoplasms - Fibroelastoma
Mediastinal Neoplasms - Fibroma
Mediastinal Neoplasms - Fibrosarcoma
Mediastinal Neoplasms - Ganglioneuroblastoma
Mediastinal Neoplasms - Ganglioneuroma
Mediastinal Neoplasms - Granular Cell Myoblastomas
Mediastinal Neoplasms - Hemangioendothelioma
Mediastinal Neoplasms - Hemangioma
Mediastinal Neoplasms, Histiocytic Lymphoma
Mediastinal Neoplasms, Immature Teratomas
Mediastinal Neoplasms, Leiomyoma
Mediastinal Neoplasms, Leiomyosarcoma
Mediastinal Neoplasms, Lipoma
Mediastinal Neoplasms, Liposarcoma
Mediastinal Neoplasms, Lymphangioma
Mediastinal Neoplasms, Lymphangiomyoma
Mediastinal Neoplasms, Malignant Fibrous Histiocytoma
Mediastinal Neoplasms, Mesenchymoma
Mediastinal Neoplasms, Mixed Embryonal Cell Carcinoma,
Mediastinal Neoplasms, Mixed Embryonal Cell Carcinoma, with
Endodermal Sinus Elements
Mediastinal Neoplasms, Mixed Embryonal Cell Carcinoma, with
Seminomatous Elements
Mediastinal Neoplasms, Mixed Embryonal Cell Carcinoma, with
Tetroid Elements
Mediastinal Neoplasms, Mixed Embryonal Cell Carcinoma, with
Trophoblastic Elements
Mediastinal Neoplasms, Myxoma
Mediastinal Neoplasms, Neuroblastoma
Mediastinal Neoplasms, Neurofibroma
Mediastinal Neoplasms, Neuroilemoma (schwannoma)
Mediastinal Neoplasms, Neurosarcoma
Mediastinal Neoplasms, Nonseminomatous
Mediastinal Neoplasms, Permeative Neuroectodermal Tumor
Mediastinal Neoplasms, Pheochromocytoma
Mediastinal Neoplasms, Pure Embryonal Cell Carcinoma
Mediastinal Neoplasms, Rhabdomyosarcoma
Mediastinal Neoplasms, Seminoma
Mediastinal Neoplasms, Xanthogranuloma
Medulary Thyroid Carcinoma
Medulloblastoma
Melanomas including:
Melanomas, Amelanotic melanoma
Melanomas, Cutaneous
Melanomas, Intraocular
Melanomas, Uveal-spindle cell (A, B, or both), mixed cell, epithelioid cell
Melanomas, Soft Tissue
Melanomas, Extraocular Extension
Melanomas, Multiple Choroidal Malignant Melanoma
Melanomas, with Brain Metastases
Melanomas, with Liver Metastases
Melanomas, with Lung Metastases
Melanomas, Melanoma-associated retinopathy
Meningeal Cancer
Meningeal Metastases
Meningioma
Merkel Cell Carcinoma
Merkel Cell Skin Cancer
Mesothelioma:
Mesothelioma, Biphasic
Mesothelioma, Epithelial
Mesothelioma, Poorly Differentiated
Mesothelioma, Sarcomatoid
Mesenchymal Chondrosarcoma
Metastatic carcinoma of unknown primary site:
Metastatic carcinoma of unknown primary site, poorly differentiated
neoplasm
Metastatic carcinoma of unknown primary site, poorly differentiated
carcinoma (with or without features of adenocarcinoma
Metastatic carcinoma of unknown primary site, well-differentiated and
moderately well-differentiated adenocarcinoma
Metastatic carcinoma of unknown primary site, Metastatic gestational
Choriocarcinoma
Metastatic Melanoma
Mixed Mullerian Tumor
Mucinous Carcinoma
Multiple Myeloma
Muscle-Invasive Bladder Tumor
Mycosis Fungoides
Myeloblastomas (chloromas or granulocytic sarcomas)
Myelodysplasia
Myelodysplastic Syndrome
Myeloma, Bence-Jones
Myeloproliferative Disorder
Myelodysplastic syndromes (MDS):
Myelodysplastic syndromes (MDS), refractory anemia (RA),
Myelodysplastic syndromes (MDS), preleukemia, or smoldering leukemia
Myelodysplastic syndromes (MDS), hypocellular MDS
Myelodysplastic syndromes (MDS), with extensive fibrosis
Myelodysplastic syndromes (MDS), unclassifiable MDS
Myelodysplastic syndromes (MDS), 5q-syndrome
Myelodysplastic syndromes (MDS), with Myelofibrosis
Myelodysplastic Syndromes (MDS), hypoplastic
Myelodysplastic Syndromes (MDS), with Eosinophilia or Basophilia
Nail/Nail Bed Cancer
Nasal Cavity Cancer
Nasopharynx Cancer
Neck, Radical Dissection
Neoplastic Meningitis
Nerve Sheath Tumors
Neuroblastoma (Adult)
Neuroblastoma (Child)
Neuroendocrine Tumors:
Neuroendocrine Tumors, Carcinoids (Foregut, Hindgut, Midgut)
Neuroendocrine Tumors, Glandular
Neuroendocrine Tumors, Insular
Neuroendocrine Tumors, Mixed
Neuroendocrine Tumors, Trabecular
Neuroendocrine Tumors, Undifferentiated
Neurofibromatosis Type I (Von Reckling-Hausen's Disease)
Neurologic Paraneoplastic Syndrome
Nose Tumor
Ocular Melanoma
Olfactory Cancer
Oligodendroglioma
Optic Glioma
Oral Cancer
Orbital Lymphoma
Oropharynx Cancer
Osteochondroma
Osteogenic Sarcoma
Osteoid Tumors
Osteosarcoma:
Osteosarcoma, Central Medullary
Osteosarcoma, Low-Grade Intraosseous
Osteosarcoma, Paget's Sarcoma
Osteosarcoma, Parosteal
Osteosarcoma, Periosteal
Osteosarcoma, Small Cell
Ovarian Cancer (Adult)
Ovarian Cancer (Child)
Ovarian Cancer, Advanced
Ovarian Cancer, Androblastoma:
Ovarian Cancer, Androblastoma, Gynandroblastoma
Ovarian Cancer, Androblastoma, Hilus Cell Tumor
Ovarian Cancer, Androblastoma, Sertolid Cell Tumor (Tubular Adenoma
of Pick)
Ovarian Cancer, Androblastoma, Sertoli-Leydig Cell Tumor
Ovarian Cancer, Androblastoma, Leydig Cell Tumor
Ovarian Cancer, Androblastoma, Tubular Androblastoma
Ovarian Cancer, Androblastoma, Unclassified
Ovarian Cancer, Borderline:
Ovarian Cancer, Borderline, Endometrioid
Ovarian Cancer, Borderline, Mucinous,
Ovarian Cancer, Borderline, Mucinous, Micropapillary Serous
Carcinoma
Ovarian Cancer, Borderline, Mucinous, Pseudomyxoma Peritonei Serous
Ovarian Cancer, Carcinosarcoma
Ovarian Cancer, Endometrioid Tumor:
Ovarian Cancer, Endometrioid Tumor, Brenner Tumor
Ovarian Cancer, Endometrioid Tumor, Carcinoma
Ovarian Cancer, Endometrioid Tumor, Carcinoma, Adenoacanthoma
Ovarian Cancer, Endometrioid Tumor, Carcinoma, Adenocarcinoma
Ovarian Cancer, Endometrioid Tumor, Carcinoma, Malignant
Adenofibroma,
Ovarian Cancer, Endometrioid Tumor, Carcinoma, Malignant
Adenofibroma,
Ovarian Cancer, Endometrioid Tumor, Carcinoma, Cystadenofibroma
Ovarian Cancer, Endometrioid Tumor, Clear Cell (mesonephroid) Tumor,
Ovarian Cancer, Endometrioid Tumor, Clear Cell (mesonephroid) Tumor,
Adenocarcinoma
Ovarian Cancer, Endometrioid Tumor, Clear Cell (mesonephroid) Tumor,
Carcinoma
Ovarian Cancer, Endometrioid Tumor, Endometrioid Stromal Sarcoma
Ovarian Cancer, Endometrioid Tumor, Mesodermal (Mullerian) Mixed
Tumor,
Ovarian Cancer, Endometrioid Tumor, Mesodermal (Mullerian) Mixed
Tumor, Heterologous
Ovarian Cancer, Endometrioid Tumor, Mesodermal (Mullerian) Mixed
Tumor, Homologous
Ovarian Cancer, Endometrioid Tumor, Mixed Epithelial Tumor
Ovarian Cancer, Endometrioid Tumor, Unclassified
Ovarian Cancer, Endometrioid Tumor, Undifferentiated Carcinoma
Ovarian Cancer, Epithelial
Ovarian Cancer, Germ Cell:
Ovarian Cancer, Germ Cell, Dysgerminoma
Ovarian Cancer, Germ Cell, Embryonal Carcinoma
Ovarian Cancer, Germ Cell, Endodermal Sinus Tumor
Ovarian Cancer, Germ Cell, Polyembryoma
Ovarian Cancer, Gonadoblastoma:
Ovarian Cancer, Gonadoblastoma, Mixed
Ovarian Cancer, Gonadoblastoma, Pure
Ovarian Cancer, Granulosa-Stromal Cell Tumor:
Ovarian Cancer, Granulosa-Stromal Cell Tumor, Fibroma
Ovarian Cancer, Granulosa-Stromal Cell Tumor, Granulosa Cell Tumor
Ovarian Cancer, Granulosa-Stromal Cell Tumor, Thecoma-Fibroma
Tumor
Ovarian Cancer, Granulosa-Stromal Cell Tumor, Unclassified
Ovarian Cancer, Lipoid Cell Tumor:
Ovarian Cancer, Lipoid Cell Tumor, Choriocarcinoma
Ovarian Cancer, Lipoid Cell Tumor, Mixed
Ovarian Cancer, Lipoid Cell Tumor, Teratoma,
Ovarian Cancer, Lipoid Cell Tumor, Teratoma, Carcinoid
Ovarian Cancer, Lipoid Cell Tumor, Teratoma, Immature
Ovarian Cancer, Lipoid Cell Tumor, Teratoma, Mature Dermoid Cyst with
Malignant Transformation
Ovarian Cancer, Lipoid Cell Tumor, Teratoma, Monodermal
Ovarian Cancer, Lipoid Cell Tumor, Teratoma, Struma Ovarii
Ovarian Cancer, Lipoid Cell Tumor, Teratoma, Struma Ovarii and
Carcinoid
Ovarian Cancer, Malignant Mucinous Tumor:
Ovarian Cancer, Malignant Mucinous Tumor, Adenocarcinoma
Ovarian Cancer, Malignant Mucinous Tumor, Adenocarcinoma
Cystadenocarcinoma
Ovarian Cancer, Malignant Mucinous Tumor, Malignant Adenofibroma
Ovarian Cancer, Malignant Mucinous Tumor, Malignant Adenofibroma
Cystadenofibroma
Ovarian Cancer, Malignant Serous Tumor:
Ovarian Cancer, Malignant Serous Tumor, Adenocarcinoma,
Ovarian Cancer, Malignant Serous Tumor, Adenocarcinoma, Papillary
Adenocarcinoma
Ovarian Cancer, Malignant Serous Tumor, Adenocarcinoma, Papillary
Cystadenocarcinoma
Ovarian Cancer, Malignant Serous Tumor, Malignant Adenofibroma,
Ovarian Cancer, Malignant Serous Tumor, Malignant Adenofibroma,
Cystadenofibroma
Ovarian Cancer, Malignant Serous Tumor, Surface Papillary Carcinoma
Ovarian Cancer, Peritoneal
Ovarian Cancer, Recurrent
Ovarian Cancer, Sarcoma
Ovarian Cancer, Stage I
Ovarian Cancer, Stage II
Ovarian Cancer, Stage III
Ovarian Cancer, Stage IV
Ovarian Cancer, Stromal Cell
Ovarian Cancer with Bone Metastases
Ovarian Cancer with Liver Metastases
Ovarian Cancer with Lung Metastases
Ovarian Cancer with Meningeal Metastases
Ovarian Cancer with Pleural Effusion
Ovarian Cancer with Spleen Metastases
Paget's Disease of the Breast
Pancreatic Cancer including:
Pancreatic Cancer, Acinar cell carcinoma
Pancreatic Cancer, Adenocarcinoma:
Pancreatic Cancer - Adenocarcinoma, Anaplastic
Pancreatic Cancer - Adenocarcinoma, Adenosquamous
Pancreatic Cancer - Adenocarcinoma, Colloid Ductal (mucinous
noncystics)
Pancreatic Cancer - Adenocarcinoma, Ductal Adenocarcinoma
Pancreatic Cancer - Adenocarcinoma, Medullary
Pancreatic Cancer - Adenocarcinoma, Signet-ring Cell
Pancreatic Cancer - Adenocarcinoma, Undifferentiated carcinoma with
osteoclast-like giant cells
Pancreatic Cancer- Carcinoid
Pancreatic Cancer, Cystic Neoplasms:
Pancreatic Cancer, Cystic Neoplasms, Intraductal Papillary Mucinous
Neoplasms
Pancreatic Cancer, Cystic Neoplasms, Mucinous Cystic Neoplasms
Pancreatic Cancer, Cystic Neoplasms, Mucinous Cystic Neoplasms
Borderline
Pancreatic Cancer, Cystic Neoplasms, Mucinous Cystic Neoplasms
Cystadenocarcinoma
Pancreatic Cancer, Cystic Neoplasms, Mucinous Cystic Neoplasms
Cystadenoma
Pancreatic Cancer, Cystic Neoplasms, Mucinous Cystic Neoplasms
Mucinous Cystic Neoplasm with In Situ Carcinoma
Pancreatic Cancer, Cystic Neoplasms, Serous Cyctic Neoplasms
Pancreatic Cancer, Inoperable
Pancreatic Cancer, Islet Cell
Pancreatic Cancer, Islet Cell, gastrinoma
Pancreatic Cancer, Islet Cell, insulinoma
Pancreatic Cancer, Islet Cell, lucagonoma
Pancreatic Cancer - Operable
Pancreatic Cancer - Pancreatoblastoma
Pancreatic Cancer with Liver Metastases
Pancreatic Cancer - Stage I
Pancreatic Cancer - Stage II
Pancreatic Cancer - Stage III
Pancreatic Cancer - Stage IVA
Pancreatic Cancer - Stage IVB
Pancreatic Cancer - Pancreatic endocrine tumors (PET)
Pancreatic Cancer - PPoma
Pancreatic Cancer - VIPoma
Pancreatic Cancer, Recurrent Pancreactic Cancer
Pancreatic Cancer, Islet cell tumors, gastrinoma
Pancreatic Cancer, Borderline Pancreatic Tumors
Paraganglioma
Paranasal Sinus Cancer
Paraneoplastic Syndromes
Ectopic Adrenocorticotropic Hormone Syndrome:
Syndrome of Inappropriate Antidiuretic Hormone Production
Protein-Losing Enteropathy
Cutaneous paraneoplastic syndrom
Paget's disease of the breast Pancreatic Cancer, Islet Cell
Acrokeratosis paraneoplastica
Melanosis
acanthosis nigricans
Sweet's syndrome
Necrolytic migratory erythema
Multicentric reticulohistiocytosis
Paraneoplastic pemphigus
Acquired ichthyosis
Dermatomyositis
Pachydermoperiostosis
Hypertrichosis lanuginosa acquisita (malignant down)
Muir-Torre syndrome
Subacute Sensory Neuronopathy and Encephalomyeloneuritis
Limbic Encephalitis
Acute polyradiculoneuropathy
Hypertrophic Osteoarthropathy
Dermatomyositis
Paraneoplastic Peripheral Neuropathies
Paraneoplastic Motor Neuron Disorders
Paraneoplastic Vision Loss
Neuromuscular Junction Disorders
Paraneoplastic neuromyotonia
paraneoplastic encephalomyelitis
Paraneoplastic parkinsonian syndromes
Parathyroid neoplasia
Parathyroid carcinoma
Parotid Gland Cancer
Pelvic Tumor
Penis Cancer:
Penis Cancer - Advanced Penile Cancer
Penis Cancer - Balantitis Xerotica Obliterans
Penis Cancer - Buschke-Löwenstein Tumor
Penis Cancer - Carcinoma In Situ
Penis Cancer - Invasive Penile Cancer
Penis Cancer - Penile Verrucous Carcinoma
Periampullary Cancer
Pericardial Effusion:
Pericardial Mesothelioma
Peripheral Nerve Sheath Tumor
Peripheral T-Cell Lymphoma
Peritoneal cancer:
Disseminated peritoneal adenomucinosis
Extraovarian Serous Carcinoma
Extraovarian Peritoneal Serous Papillary Carcinoma
Serous Surface Carcinoma of the Peritoneum
Serous Surface Papillary Carcinoma
Papillary Serous Carcinoma of the Peritoneum
Multiple Focal Peritoneal Papillary Carcinoma
Primary peritoneal carcinoma
Primary Peritoneal Mesothelioma:
Adenomatoid
localized fibrous mesothelioma
multicystic and well-differentiated papillary peritoneal mesotheliomas
epithelial
sarcomatoid
mixed (elements of both epithelial and sarcomatoid
Primary Peritoneal Papillary Serous Adenocarcinoma
Peritoneal Carcinomatosis:
mucinous adenocarcinoma of the appendix and colon
adenocarcinoma of the colon, stomach, or pancreas
Pseudomyxoma Peritonei
Peutz-Jeghers syndrome
Pheochromocytoma
Pinealoma
Pineoblastoma
Placental-Site Trophoblastic Tumors
Plasmacytoma
Plasma Cell Neoplasms:
plasma cell myeloma
monoclonal gammopathy of unknown significance (MGUS)
astleman's disease
α-heavy-chain disease
Waldenström's macroglobulinemia (WM)
plasma cell leukemia
multiple myeloma (MM)
Solitary Plasmacytoma
Indolent MyelomaPleura Cancer
Pleura Metastases
Pleural Effusion, Malignant
Pleural Mesothelioma, Malignant
Preleukemia
Primary Central Nervous System Lymphoma
Primitive Neuroectodermal Tumor (PNET) (Adult)
Primitive Neuroectodermal Tumor (PNET) (Child)
Prolymphocytic Leukemia
Promyelocytic Leukemia
Prophylactic Mastectomy
Prostate Cancer:
Acinar Adenocarcinoma
Ductal Carcinoma
Intralobular Acinar Carcinoma
Malignant Mesenchymal Tmors
Angiosarcoma
Chondrosarcoma
Liposarcoma
Myosarcoma
Osteosarcoma
Rhabdomyosarcomas
Leiomyosarcomas
Mucinous Carcinoma
Proximal Ductal Epithelium Adenocarcinoma
Scirhous Patern Tumors
Transitional Cell Tumors
Prostate Cancer, Early-Stage
Prostate Cancer, Hormone Refractory
Prostate Cancer in the Elderly
Prostate Cancer, Small Cell
Prostate Cancer, Stage A (Stage I)
Prostate Cancer, Stage B (Stage II)
Prostate Cancer, Stage C (Stage III)
Prostate Cancer, Stage D (Stage IV)
Prostate Cancer, Transitional Cell
Prostate Cancer with Bone Metastases
Prostatic Intraepithelial Neoplasia (PIN)
PSA
Pseudomyxoma Peritonei
Rectal Cancer, Stage I
Rectal Cancer, Stage II
Rectal Cancer, Stage III
Rectal Cancer with Brain Metastases
Rectal Cancer with Liver Metastases
Recurrent Colon Cancer
Recurrent Colorectal Cancer
Recurrent Lung Cancer
Recurrent Melanoma
Recurrent Ovarian Cancer
Recurrent Prostate Cancer
Recurrent Rectal Cancer
Relapsed Lymphoma
Renal Cell Carcinoma
Renal Cell Carcinoma with Lung Metastases
Renal Pelvis Cancer
Retinoblastoma (Adult)
Retinoblastoma (Child)
Rhabdomyosarcoma
Richter's Syndrome
Rising PSA as an Indication of Recurrent Prostate Cancer
Salivary Gland Cancer including:
Adenoid cystic carcinoma
Mucoepidermoid carcinoma
Sarcoma of Bone
Sarcomatoid Carcinoma
Schwannoma, Malignant
Sebaceous Carcinoma
Sezary Syndrome
Sinus, Cavernous
Sinus Cancer
Sinus Cancer, Cavernous
Sinus Cancer, Endodermal
Sinus Cancer, Esthesioneuroblastoma
Sinus Cavity Cancer
Skin Cancer:
Basal Cell- nodular, nevoid, superficial, morpheaform (also termed
aggressive-growth BCC or infiltrative BCC), pigmented, cystic BCC, and
fibroepithelioma of Pinkus (FEP)
Merkel Cell
Squamous Cell- keratoacanthoma, periungual
Skin Metastases
Atypical fibroxanthoma
Actinic keratoses
Dermatofibroma
Desmoplastic trichoepithelioma
Microcystic adrexal carcinoma
Non-melanoma skin cancer
Verrucous carcinoma
Malignant Fibrous Histiocytoma
Dermatofibrosarcoma Protuberans
Bednar Tumor
Angiosarcoma
Kaposi's Sarcoma
Carcinoma Metastatic to Skin
Small Cell Bladder Cancer
Small Cell Lung Cancer
Small Cell Lung Cancer, Extensive Stage
Small Cell Lung Cancer, Limited Stage
Small Cell Lung Cancer with Brain Metastases
Small Cell Lung Cancer with Liver Metastases
Small Cleaved Cell Lymphoma
Small Intestine Cancer, including:
Adenocarcinoma
Carcinoid
Foregut
Hindgut
Midgut
Fibromatosis (Desmoid Tumor)
Gastrointestinal Stromal Tumor
Inflammatory Fibroid Polyps
Lymphoma
Burkitt's Lymphoma
Diffuse Large B-Cell Lymphoma
Mucosal-Associated Lymphoid Tissue Lymphoma
T-Cell Lymphomas of the Small Intestine
Myofibroblastic Tumor
Schwannoma
Simple Leiomyoma
Solitary Fibrous Tumor
True Leiomyoma
Soft Tissue Lymphoma
Soft Tissue Sarcoma:
Soft Tissue Sarcoma (Adult)
Soft Tissue Sarcoma (Child)
Soft Tissue Sarcoma, Angiosarcoma
Soft Tissue Sarcoma, Clear Cell
Soft Tissue Sarcoma, Dermatofibrosarcoma Protuberans
Soft Tissue Sarcoma, Hemangiopericytoma
Soft Tissue Sarcoma, Leiomyosarcoma
Soft Tissue Sarcoma, Liposarcoma
Soft Tissue Sarcoma, Malignant Fibrous Histiocytoma
Soft Tissue Sarcoma, Primitive Neuroectodermal (Adult)
Soft Tissue Sarcoma, Primitive Neuroectodermal (Child)
Soft Tissue Sarcoma, Rhabdomyosarcoma
Soft Tissue Sarcoma, Synovial
Synovial Sarcoma
Epithelioid Sarcoma:
Ewing's Sarcoma
Extraskeletal Cartilaginous Tumors
Extraskeletal Chondrosarcoma
Mesenchymal Chondrosarcoma
Myxoid Chondrosarcoma
Extraskeletal Osteosarcoma
Fibrohistiocytic Tumors
Atypical Fibroxanthoma
Angiomatoid Fibrous Histiocytoma
Dermatofibrosarcoma Protuberans
Giant Cell Fibroblastoma
Malignant Fibrous Histiocytoma
Giant Cell
Inflammatory
Myxoid
Storiform-Pleomorphic
Plexiform Fibrohistiocytic Tymor
Fibrous Tumors
Fibromatoses
Deep Fibromatoses
Abdominal Fibromatosis
Extraabdominal Fibromatosis
Intraabdominal Fibromatosis
Infantile Fibromatosis
Mesenteric Fibromatosis
Superficial Fibromatoses
Palmar
Penile (Peyronie's Fibromatosis)
Fibrosarcoma
Adult Fibrosarcoma
Inflammatory Fibrosarcoma
Infantile Fibrosarcoma
Inflammatory Myofibroblasic Tumor
Gastrointestinal Stromal Tumors
Kaposi's Sarcoma
Lipomatous Tumors
Atypical Lipoma
Liposarcoma
Dedifferentiated Liposarcoma
Myxoid (Round Cell) Liposarcoma
Pleomorphic Liposarcoma
Well-differentiated Liposarcoma
Inflammatory Liposarcoma
Lipoma-like Liposarcoma
Sclerosing Liposarcoma
Lymphangiosarcoma
Malignant Giant Cell Tumor of Tendon Sheath
Malignant Glomus Tumor (Glomangiosarcoma)
Malignant Extrarenal Rhabdoid Tumor
Malignant Hemangiopericytoma
Malignant Mesenchymoma
Neural Tumors
Malignant Peripheral Nerve Sheath Tumor (MPNST)
Malignant Triton Tumor
Epithelioid MPNST
Glandular MPNST
Reticulum Cell Sarcoma (Lymphoma of bone)
Skeletal Muscle Tumors
Ectomesenchymoma
Rhabdomyosarcoma
Alveolar Rhabdomyosarcoma
Botryoid Rhabdomyosarcoma
Embryonal Rhabdomyosarcoma
Pleomorphic Rhabdomyosarcoma
Spindle Cell Rhabdomyosarcoma
Small Round Cell Sarcoma
Histiocytosis
Metastatic Neuroblastoma
Metastatic Undifferentiated Carcinoma
Multiple Myeloma
Osteomyelitis
Small Cell Osteosarcoma
Smooth Muscle Tumors
Epithelioid Leiomyosarcoma
Leiomyosarcoma
Solitary Fibrous Tumor
Spindle Cell Hemangioendothelioma
Synovial Sarcoma
Biphasic Synovial Sarcoma
Monophasic Synovial Sarcoma
Spinal Cord Cancer
Spindle Cell Thyroid Cancer
Squamous Cell Cancer (Non-Skin)
Squamous Cell Lung Cancer
Squamous Cell Skin Cancer
Stem Cell Transplantation
Stomach Cancer, including:
Stomach Cancer, Adenocanthoma
Stomach Cancer, Adenocarcinoma
Stomach Cancer, Adenosquamous Carcinoma
Stomach Cancer, Gastric Carcinoid
Stomach Cancer, Gastric Leiomyosarcoma
Stomach Cancer, Gastric Lymphoma
Stomach Cancer, Gastric Plasmacytoma
Stomach Cancer, Linitis Plastica
Stomach Cancer, Scirrhous Carcinoma
Stomach Cancer, Signet Ring Cell
Stomach Cancer, Mucinous Adenocarcinoma
Stomach Cancer, Mixed adeno- and Choriocarcinoma
Stomach Cancer, Papillary Adenocarcinoma
Stomach Cancer, Squamous Cell Carcinoma
Stomach Cancer with Bone Metastases
Superficial Bladder Cancer
Sweat Glands Cancer
T-Cell Leukemia
T-Cell Lymphoma, Cutaneous
Testicular Cancer, Adenocarcinoma of the Rete Testis
Testicular Cancer, Carcinoma In Situ
Testicular Cancer, Choriocarcinoma
Testicular Cancer, Epidermoid Cyst
Testicular Cancer, Germ Cell
Testicular Cancer, Gonadoblastoma
Testicular Cancer, Granulosa Cell Tumors
Testicular Cancer, Leydig Cell Tumors
Testicular Cancer, Lymphoma
Testicular Cancer, Mesothelioma
Testicular Cancer, Metastatic Carcinoma
Testicular Cancer, Non-Seminoma:
Choriocarcinoma
Embryonal Carcinoma
Teratoma
Immature
Mature
With Malignant Differentiation
Yolk Sac
Testicular Cancer, Seminoma:
Anaplastic
Atypical
Typical
Testicular Cancer, Sarcomas
Testicular Cancer, Sertoli Cell Tumors:
Classic
Large Cell Calcifying
Sclerosing
Testicular Cancer, Stage I
Testicular Cancer, Stage II
Testicular Cancer, Stage III
Thalamus (Thalamic) Brain Tumor
Thymoma
Thymus Cancer, including:
Thymic Carcinoid
Thymic Carcinoma
Clear-Cell Carcinoma
Lymphoepithelioma-like Carcinoma
Mucoepidermoid Carcinoma
Sarcomatoid
Squamous Cell Carcinoma
Thymolipoma
Thymoma
Cortical
Medullary
Mixed
Thyroid Cancer:
Papillary thyroid carcinoma with papillary renal neoplasia (PTC-RCC)
Familial nonmedullary thyroid carcinoma (fNMTC)
Familial thyroid tumors with cell oxyphilia (TCO)
Papillary thyroid cancer without oxyphilia
Familial adenomatous polyposis (FAP)
Thyroid malignancy derived from follicular or parafollicular C cells
Medullary thyroid carcinoma (MTC)
Cowden disease
Thyroid Lymphoma
Tongue Cancer
Tongue Base Cancer
Transitional Cell Cencer of Bladder
Transitional Cell Cancer of Renal Pelvis
Transitional Cell Cancer of the Ureter
Transition Cell Cancer of the Urethra
Tuberous Sclerosis (Bourneville Disease)
Tylosis
Unknown Primary Cancer
Ureter Cancer
Urethra Cancer:
Adenocarcinoma
Clear Cell
Melanoma
Squamous Cell
Transitional Cell
Undifferentiated
Urethra Cancer, Carcinoma of Bulbomembranous Urethra
Urethra Cancer, Carcinoma of Distal Urethra
Urethra Cancer, Carcinoma of Prostatic Urethra
Urethra Cancer, Carcinoma of Proximal Urethra
Uterine Cancer
Uterine Cancer, Endometrial
Uterine Cancer, Carcinosarcoma
Uterine Cancer, Leiomyosarcoma
Uterine Cancer, Papillary Serous
Uterine Cancer, Sarcoma
Uterine Cancer, Stage I
Uterine Cancer, Stage II
Uterine Cancer, Stage III
Uterine Cancer, Stage IV
Uterine Sarcoma
Vagina Cancer:
Adenocarcinomas
Adenosquamous
Clear Cell
Mucinous
Papillary
Undifferentiated
Primary Vaginal Melanomas
Sarcoma
Embryonal Rhabdomyosarcoma (Sarcoma Botryoides)
Endometrial Stromal Sarcoma
Leiomyosarcoma
Malignant Mixed Mullerian Tumor
Small Cell Carcinoma
Squamous Cell Carcinoma
Keratinizing
Nonkeratinizing
Vaginal Intraepithelial Neoplasia
Verrucous Carcinoma
Vocal Cord Cancer
Vulva Cancer:
Adenocarcinoma
Anaplastic Carcinomas
Large Immature Cells
Spindle Sarcomatoid Cells
Small Cells
Bartholin's Gland Carcinoma
Adenocarcinoma
Adenoid Cystic Carcinoma
Squamous Cell Carcinoma
Transitional Cell Carcinoma
Basal Cell Carcinoma
Lichen Sclerosis
Malignant Melanoma
Paget's Lesions
Primary Mammary Adenocarcinoma
Sebaceous Carcinoma
Squamous Cell Carcinoma
Squamous Hyperplasia
Verrucous Carcinoma
Vulvar Intraepithelial Neoplasia
Vulvar Sarcoma
Angiosarcoma
Epithelioid Sarcoma
Leiomyosarcoma
Neurofibrosarcoma
Rhabdomyosarcoma
Waldenstrom's Macroglobulinemia
Warthin's Tumor
Wilms' Tumor (Child)
Zollinger-Ellison Syndrome

2. A method of claim 1, wherein said cancer is Adenocarcinoma.

3. A method of claim 1, wherein said cancer is adenocarcinoma of the colon, stomach, or pancreas.

4. A method of claim 1, wherein said cancer is Kidney Cancer (Renal Cell Carcinoma)—Clear Cell Renal Carcinoma.

5. A method of claim 1, wherein said cancer is Liver Cancer—Hepatocellular Carcinoma (Liver Cell Carcinoma).

6. A method of claim 1, wherein said cancer is Lung Cancer—Adenocarcinoma.

7. A method of claim 1, wherein said cancer is Stomach Cancer—Adenocarcinoma.

8. A method of claim 1, wherein said cancer is Ovarian Cancer, Carcinosarcoma.

9. A method of claim 1, wherein said cancer is Ovarian Cancer (Adult).

10. A method of claim 1, wherein said cancer is Adenocarcinoma Lung Cancer.

11. A method of claim 1, wherein said cancer is metastatic breast cancer.
